(19) United States
(12) Patent Application Publication
(10) Pub. No.: US 2005/0233346 A1
(43) Pub. Date: Oct. 20, 2005

(54) Plant promoters and methods of use thereof

(75) Inventors: Richard A. Dixon, Ardmore, OK (US); Liangjiang Wang, Ardmore, OK (US); Gregory D. May, Ardmore, OK (US)

Correspondence Address: FULBRIGHT & JAWORSKI L.L.P., 600 CONGRESS AVE., SUITE 2400, AUSTIN, TX, 78701, US

(73) Assignee: The Samuel Robert Noble Foundation

(21) Appl. No.: 10/955,924

(22) Filed: Sep. 30, 2004

Related U.S. Application Data
(60) Provisional application No. 60/507,362, filed on Sep. 30, 2003.

(51) Int. Cl. C12Q 1/68; A01H 1/00

(52) U.S. Cl. 435/6

(57) ABSTRACT

The current invention provides plant promoter sequences. Compositions comprising these promoter sequence are described, as are plants transformed with such compositions. Further provided are methods for the expression of transgenes in plants comprising the use of these sequences. The methods of the invention include the direct creation of transgenic plants with the promoters by genetic transformation, as well as by plant breeding methods. The sequences of the invention represent valuable new tools for the creation of transgenic plants, preferably having one or more added beneficial characteristics.

This application claims the opriority of U.S. Provisional Patent Appl. Ser. No. 60/507,362, filed Sep. 30, 2003, the entire disclosure of which is specifically incorporated herein by reference.

### BACKGROUND OF THE INVENTION

I. Field of the Invention

The present invention generally relates to new regulatory sequences with defined tissue specificity. More specifically, the invention relates to the discovery of gene promoters for constitutive and inducible expression of transgenes for the improvement of plant species.

II. Description of Related Art

Most of the research funding for plant improvement in the United States has, historically, gone to the major commodity crops such as corn, wheat and soybean. More recently, the worldwide revolution in plant genomics has been centered primarily with just two species, Arabidopsis thaliana and rice (Bevan et al., 1999; Delseny et al., 2001; Ausubel, 2002; Goff et al., 2002). A vigorous debate continues as to how the findings made in these model systems will translate to other economically important species.

Molecular improvement of forage crops presents both new challenges and clear opportunities for the application of biotechnology. In the past several years, researchers worldwide have begun to develop genetic model systems for forage legumes and grasses. Forage quality traits such as digestibility, nutritional quality and palatability present the molecular biologist with interesting new targets for gene discovery. Genetic modification of these traits should enhance economics, animal health and the environment, and presents a case study for explaining the potential benefits of GMOs. Many of these studies have used previously available constitutive promoters such as the cauliflower mosaic virus ³⁵S (CaMV35S) promoter. However, the ³⁵S promoter does not function well and reproducibility in all plant species. There is a very clear need for gene promoters (regulatory sequences) for the tissue-specific and inducible expression of a variety of transgenes useful for plants and particularly forage species, including forage legumes, of which the major example is alfalfa (Medicago sativa).

Classical breeding approaches have, with few exceptions, been the mainstay of forage improvement over the past 50 years or more. More recently, molecular tools such as QTL analysis and marker assisted selection have facilitated these endeavors. Knowing the functions of all the genes within a plant would provide an invaluable resource for molecular breeding. However, the genomes of most forage crops (grasses and legumes) are complex and unlikely to be subjects of large scale sequencing projects in the foreseeable future. Major advances in forage improvement may come from transgenic approaches utilizing transgenes discovered in model species.

With more than 18,000 species, members of the pea family (Leguminosae) are second only to grasses in economic importance worldwide. Forage and pasture legumes are an important source of nutrition for animal and dairy production. Seed legumes such as peanut, soybeans, chickpeas, and lentils are from 20 to 50 percent protein—two to three times that of cereal grains and meat. Legumes therefore serve as an excellent source of protein and dietary fiber that is often deficient in the diets of individuals in developing nations. Moreover, in comparison with other crops, the production of legumes reduces economic and environmental costs given their ability to fix nitrogen.

Medicago truncatula, also known as barrel medic because of the shape of its seed pods, is a forage legume commonly grown in Australia. It originates from Mediterranean regions, and has recently been introduced as a warm season annual legume to the Gulf Coast States in the US. M. truncatula is very closely related to the world's major forage legume, alfalfa (Medicago sativa). However, whereas alfalfa is an outcrossing autotetraploid with four copies of each of its eight chromosomes, M. truncatula has a simple diploid genome and can be readily self-pollinated, facilitating genetic analysis.

In addition to its small genome, M. truncatula has a fast generation time and can be transformed genetically using relatively standard protocols, and has thus been adopted as a model for legume genomics (Cook, 1999; Oldroyd and Geurts, 2001). Genes from M. truncatula share very high sequence identity to their counterparts from alfalfa, and also appear to be arranged in a similar order on the chromosomes, making M. truncatula an excellent model for understanding the molecular biology of alfalfa.

The Medicago gene index at the National Center for Genome Resources (Bell et al., 2001) and the TIGR Medicago gene index (Quackenbush et al., 2000), provide information on approximately 200,000 expressed sequence tags (ESTs) from M. truncatula, and a whole genome sequencing project for M. truncatula is in progress at the University of Oklahoma. It is possible to view and analyze sequences of all the expressed genes sequenced to date, and to compute their expression patterns in silico, by simple search and query commands with various Plant Gene Index databases, such as those available at the TIGR website (http://www.tigr.org/tdb/tgi.shtml). An important feature of the M. truncatula EST data is that nearly 40 different cDNA libraries, representing a range of tissues and biological conditions, have been sequenced, greatly facilitating in silico analysis of gene expression patterns.

Although previous studies have provided a number of useful tools for the generation of transgenic plants, there is still a great need in the art for novel promoter sequences with beneficial expression characteristics. The number of effective promoters available for use with transgenes in plants is not abundant. New promoters, especially promoters that have different expression profiles are needed. Availability of a wider range of effective promoters would make it possible to introduce multiple transgenes into a plant, each fused to a different promoter, thereby minimizing the risk of DNA sequence homology dependent transgene inactivation (co-suppression). Therefore, there is a great need in the art for the identification of novel promoters with differing expression profiles that may be used for the beneficial expression of selected transgenes in economically important crop plants.

### SUMMARY OF THE INVENTION

In one aspect, the invention provides an isolated nucleic acid sequence comprising a promoter sequence operable in a plant, wherein the promoter comprises a nucleic acid sequence selected from the group consisting of SEQ ID NO:1 through SEQ ID NO:26; or a fragment thereof having promoter activity. In certain embodiments, the fragment may be further defined as comprising at least 25, 50, 75, 95, 125, 250, 500 or more contiguous nucleotides of any one of the sequences in SEQ ID NO:1 through SEQ ID NO:26, up to and including the full length sequence. The isolated nucleic acid sequence may further be defined as operably linked to any desired elements, including an enhancer and/or coding sequence.

In yet another aspect, the invention provides a transformation construct comprising: (a) an isolated nucleic acid sequence comprising a promoter sequence operable in a plant, wherein the promoter comprises a nucleic acid sequence selected from the group consisting of SEQ ID NO:1 through SEQ ID NO:26; or a fragment thereof having promoter activity; and (b) a heterologous coding sequence operably linked to said promoter sequence. The coding sequence may in certain embodiments, be operably linked to a terminator, an enhancer and/or a selectable marker. The transformation construct may further comprise at least a second promoter and/or second promoter. The transformation construct may also comprise a screenable marker.

In still yet another aspect, a plant is provided that is transformed with a selected DNA comprising a promoter sequence of a promoter sequence operable in a plant, wherein the promoter comprises a nucleic acid sequence selected from the group consisting of SEQ ID NO:1 through SEQ ID NO:26; or a fragment thereof having promoter activity. The plant may be further defined as a dicotyledonous plant or monocotyledonous plant.

In certain other embodiments of the invention, nucleic acids hybridizing to any one of SEQ ID NO:1 through SEQ ID NO:26 under stringent conditions and having promoter activity are provided. Stringent conditions may be defined as 5×SSC, 50% formamide and 42° C. By conducting a wash under such conditions, for example, for 10 minutes, those sequences not hybridizing to a particular target sequence under these conditions can be removed. A sequence provided by the invention may be defined as a sequence having at least 95% sequence identity to a nucleic acid sequence selected from any one or more of SEQ ID NO:1 through SEQ ID NO:26 and having promoter activity.

In still yet another aspect, a cell of a plant of the invention is provided. A seed of a plant of the invention is provided in still another embodiment. In still yet another embodiment, a progeny plant of any generation of a plant of the invention is provided, wherein the progeny plant comprises a selected DNA.

In still yet another aspect, the invention provides a method of expressing a polypeptide in a plant cell comprising the steps of: (a) obtaining a construct comprising a promoter sequence operable in a plant, wherein the promoter comprises a nucleic acid sequence selected from the group consisting of SEQ ID NO:1 through SEQ ID NO:26; or a fragment thereof having promoter activity operably linked to a heterologous coding sequence encoding a polypeptide; and (b) transforming a recipient plant cell with the construct, wherein said recipient plant cell expresses said polypeptide. In the method, the plant cell may be further defined as a dicotyledonous or monocotyledonous plant cell.

In still yet another aspect, a method of producing a plant transformed with a selected DNA comprising a promoter sequence operable in a plant is provided, wherein the promoter comprises a nucleic acid sequence selected from the group consisting of SEQ ID NO:1 through SEQ ID NO:26, or a fragment thereof having promoter activity, comprising: (a) obtaining a first plant comprising the selected DNA; (b) crossing the first plant to a second plant lacking said selected DNA; and (c) obtaining at least a first progeny plant resulting from said crossing, wherein said progeny plant has inherited said selected DNA. In the method, the plant may be further defined as a dicotyledonous plant or monocotyledonous plant.

### DETAILED DESCRIPTION OF THE INVENTION

The invention overcomes the limitations of the prior art by providing promoters for the expression of heterologous coding sequences in plants. The promoter sequences were originally identified in Medicago truncatula with a range of tissue and inducer specificities. The promoters may be used for expression of transgenes in plants and have particular benefit for legume improvement.

DNA sequence information alone is only one part of an integrated genomics program. Having a “unigene set” of all the expressed genes in a plant allows the researcher to analyze responses to biotic and abiotic stresses, and developmental programs, on a global level using DNA array techniques (Wu et al., 2001). However, such an approach is essentially correlative, and does not provide the final proof of gene function. For this reason, it is important to develop both forward and reverse genetic approaches to examine plant gene function. This has been successfully done in Arabidopsis, where T-DNA insertion mutants (Azpiroz-Leehan and Feldmann, 1997) now exist in essentially every gene in the organism, and T-DNA activation tagging can be employed to generate dominant, gain of function mutations (Weigel et al., 2000). Development of such resources, which might also include alternative mutational approaches such as transposon tagging (Fitzmaurice et al., 1992), fast neutron bombardment to generate deletions, or virus-induced gene silencing for rapid transient analysis of target gene function (Baulcombe, 1999), will be a rate limiting factor for the full exploitation of genomics approaches to forage crops. Whatever the approach taken, confirmation of gene function by stable transformation will remain an essential requirement, and this requires careful choice of gene promoters for demonstration of optimal transgene efficacy.

It is possible that knowledge derived from the Arabidopsis resources will in some cases be of value for forage crop improvement, the understanding of new pathways for lignin biosynthesis being an example (Humphreys and Chapple, 2002). Furthermore, it will not be feasible to develop genetic systems for each individual forage crop. M. truncatula provides an excellent model for alfalfa, except for the fact that it is an annual whereas alfalfa is a perennial. Among the monocots, rice has the advantage of a sequenced genome and quite good genetic resources (Ronald et al., 1992; Matsumura et al., 1999; Goff et al., 2002). Corn has had excellent genetic resources for many years (Gierl and Saedler, 1989), but has a very large genome. Comparative mapping studies (Ahn et al., 1993) should allow translation of genetic data from the more tractable systems to genetically complex forage grasses.

The targeted modification of biochemical pathways for forage crop improvement requires knowledge of the pathways themselves at the enzymatic and underlying genetic levels. Three such pathways are outlined herein as examples of the types of traits and pathways that can be modified by transgenic approaches for forage improvement. In some cases, such as that of the lignin pathway, detailed knowledge of the biochemical pathways is available, and successes have already been reported. In other cases, such as the tannins and saponins, there is still a need for basic gene discovery, making these pathways prime candidates for the genomics approach. By providing promoter elements with selected expression profiles, the invention provides important tools for these or any other desired uses.

I. Promoter Sequences and Transformation Constructs

One aspect of the current invention comprises a promoter sequence exemplified by any one of SEQ ID NO:1-SEQ ID NO:26 and fragments thereof having promoter activity. In certain embodiments of the invention, such a fragment may be a restriction fragment, for example, from a complete or partial digest by one or more restriction enzymes including, but not limited to, HaeIII, PaeI, ThaI, TrulI, TaqI, BfaI, AccI, AluI, CfoI.5, EcoRI, BamHI. Fragments may also routinely be made by mechanical shearing of DNA as well as use of non-specific nucleases. In certain aspects of the invention, a fragment of a promoter sequence having promoter activity comprises at least about 60, 80, 100, 150, 200, 300, 500 or more contiguous base pairs or more of SEQ ID NOs:1-26. In certain further embodiments of the invention, sequences are provided comprising nucleotides 1-500, 250-750, 500-1000, 500-1500, 250-1250 and/or 500-1250 of the nucleic acid sequence of any of SEQ ID NOs:1-26. By SEQ ID NO:1 through SEQ ID NO:26 it is specifically included any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20 SEQ ID NO:21, SEQ ID NO:22, SEQ ID NO:23, SEQ ID NO:24, SEQ ID NO:25 and/or SEQ ID NO:26.

The screening of promoter sequences for activity is routine in art and is carried out as described herein. For example, this can be efficiently carried out by linking promoter fragments to a screenable marker and transforming the fragments into plant cells ex vivo in transient assays for the identification of the marker gene product. This may be, for example, a visible marker. Expression of the marker indicates promoter function.

In general, the technique for creation of recombinant constructs comprising different promoter sequences are well known in the art, as exemplified by various publications. Such constructs may be engineered at the nucleotide level, for example, using site-directed mutagenesis. This is typically performed by first obtaining a single-stranded vector or melting apart of two strands of a double-stranded vector which includes a DNA sequence which comprises a promoter of the invention. An oligonucleotide primer bearing the desired mutated sequence is prepared, generally synthetically. This primer is then annealed with the single-stranded vector, and subjected to DNA polymerizing enzymes such as the E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and cells are selected which include recombinant vectors bearing the mutated sequence arrangement. Vector DNA can then be isolated from these cells and used for transformation. A genetic selection scheme was devised by Kunkel et al. (1987) to enrich for clones incorporating mutagenic oligonucleotides. Alternatively, the use of PCR™ with commercially available thermostable enzymes such as Taq polymerase may be used to incorporate a mutagenic oligonucleotide primer into an amplified DNA fragment that can then be cloned into an appropriate cloning or expression vector. The PCR™-mediated mutagenesis procedures of Tomic et al. (1990) and Upender et al. (1995) provide two examples of such protocols. A PCR™ employing a thermostable ligase in addition to a thermostable polymerase also may be used to incorporate a phosphorylated mutagenic oligonucleotide into an amplified DNA fragment that may then be cloned into an appropriate cloning or expression vector.

The term template-dependent process refers to nucleic acid synthesis of an RNA or a DNA molecule wherein the sequence of the newly synthesized strand of nucleic acid is dictated by the well-known rules of complementary base pairing (see, for example, Watson and Ramstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety. A number of template dependent processes are available to amplify the target sequences of interest present in a sample, such methods being well known in the art and specifically disclosed herein below.

An efficient, targeted means for preparing promoters relies upon the identification of putative regulatory elements within the target sequence. This can be initiated by comparison with, for example, promoter sequences known to be expressed in a similar manner. Sequences which are shared among elements with similar functions or expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter sequence is provided, any of a number of different functional deletion mutants of the starting sequence could be readily prepared.

As indicated above, fragments of a promoter also could be randomly prepared and then assayed. With this strategy, a series of constructs are prepared, each containing a different portion of the clone (a subclone), and these constructs are then screened for activity. A suitable means for screening for activity is to attach a promoter fragment to a selectable or screenable marker, and to isolate only those cells expressing the marker protein. In this way, a number of different promoter constructs are identified which still retain the desired, or even enhanced, activity. The smallest segment which is required for activity is thereby identified through comparison of the selected constructs. This segment may then be used for the construction of vectors for the expression of exogenous protein.

One important application of the promoters provided by the invention will be in the construction of vectors designed for introduction into host cells by genetic transformation. The construction of vectors which may be employed according to the invention will be known to those of skill of the art in light of the present disclosure (see for example, Sambrook et al., 2001). The techniques of the current invention are thus not limited to any particular DNA sequences in conjunction with the promoter sequences of the invention. For example, the promoters alone could be transformed into a cell with the goal of enhancing or altering the expression of one or more genes in the host genome.

Transformation vectors can be used to direct the expression of a selected coding region which encodes a particular protein or polypeptide product in a transgenic cell. In certain embodiments, a recipient cell may be transformed with more than one transformation construct. Two or more transgenes can also be introduced in a single transformation event using either distinct selected protein-encoding vectors, or using a single vector incorporating two or more gene coding sequences. Of course, any two or more transgenes of any description may be employed as desired.

Vectors used for transformation may include, for example, plasmids, cosmids, YACs (yeast artificial chromosomes), BACs (bacterial artificial chromosomes) or any other suitable cloning system, and the nucleic acids selected therefrom. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively).

Particularly useful for transformation may be expression cassette portions of vectors, isolated away from sequences not essential for expression in plants. DNA segments used for transforming cells will generally comprise the cDNA, gene or genes which one desires to introduced into and have expressed in the host cells. These DNA segments can further include, in addition to a promoter, structures such as promoters, enhancers, terminators, polylinkers, or even regulatory genes as desired. The DNA segment or gene chosen for cellular introduction may encode a protein which will be expressed in the resultant recombinant cells resulting in a screenable or selectable trait and/or which will impart an improved phenotype to the resulting transgenic cell or an organism. Alternatively, the vector may comprise a coding sequence for a protein or polypeptide which is to be isolated from the transgenic cells or is excreted from the transgenic cells. Exemplary components that may advantageously be used with transformation vectors are provided as follows.

A. Regulatory Elements

In addition to a promoter sequence, constructs prepared in accordance with the invention may comprise additional desired elements. For example, one aspect of the invention relates to the preparation of transformation constructs comprising the promoter operably linked to a selected coding region. Additionally, by including an enhancer sequence with such constructs, the expression of the selected protein may be enhanced. Enhancers often are found 5′ to the start of transcription in a promoter that functions in eukaryotic cells, but can often be inserted in the forward or reverse orientation 5′ or 3′ to the coding sequence.

Where an enhancer is used in conjunction with a promoter for the expression of a selected protein, it will often be preferable to place the enhancer between the promoter and the start codon of the selected coding region. However, one also could use a different arrangement of the enhancer relative to other sequences and potentially still realize the beneficial properties conferred by the enhancer. For example, the enhancer could be placed 5′ of the promoter region, within the promoter region, within the coding sequence (including within any intron sequences which may be present), or 3′ of the coding region.

It also is contemplated that expression of one or more transgenes may be eliminated upon induction of the promoters provided herein. In particular, by operably linking a promoter to a coding sequence in antisense orientation, accumulation of the respective protein encoded by the sense transcript could be eliminated or decreased upon expression with the promoter.

B. Terminators

Transformation constructs prepared in accordance with the invention will typically include a sequence that acts as a signal to terminate transcription and allow for the poly-adenylation of the mRNA produced by coding sequences operably linked to a promoter of the invention. The termination sequence is preferably located in the 3′ flanking sequence of a coding sequence, which will contain proper signals for transcription termination and polyadenylation. Many such terminator sequences are known to those of skill in the art. In constructing suitable expression constructs, the termination sequences associated known genes from the host organism which are efficiently expressed in particular may be ligated into the expression vector 3′ of the heterologous coding sequences to provide polyadenylation and termination of the mRNA.

C. Marker Genes

By employing a selectable or screenable marker gene as, or in addition to, a particular gene of interest, one can provide or enhance the ability to identify transformants. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening”' (e.g., the green fluorescent protein). Of course, many examples of suitable marker genes are known to the art and can be employed in the practice of the invention.

Included within the terms selectable or screenable marker genes also are genes which encode a “secretable marker” whose secretion can be detected as a means of identifying or selecting for transformed cells. Examples include marker genes which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions may be used in connection with a promoter of the present invention. Examples of selectable markers include neo (Potrykus et al., 1985), which provides kanamycin resistance and can be selected for using kanamycin, G418, paromomycin, etc.; bar, which confers bialaphos or phosphinothricin resistance; a nitrilase such as bxn from Klebsiella ozaenae which confers resistance to bromoxynil (Stalker et al., 1988) and a mutant acetolactate synthase (ALS) which confers resistance to imidazolinone, sulfonylurea or other ALS inhibiting chemicals (European Patent Application 154,204, 1985) and a methotrexate resistant DHFR (Thillet et al., 1988).

Screenable markers that may be employed include a β-glucuronidase (GUS) or uidA gene, isolated from E. coli, which encodes an enzyme for which various chromogenic substrates are known; a β-lactamase gene (Sutcliffe, 1978), which encodes an enzyme for which various chromogenic substrates are known (e.g., PADAC, a chromogenic cephalosporin); a β-galactosidase gene, which encodes an enzyme for which there are chromogenic substrates; a luciferase (lux) gene (Ow et al., 1986), which allows for bioluminescence detection or a gene encoding for green fluorescent protein (Sheen et al., 1995; Haseloff et al., 1997; Reichel et al., 1996; Tian et al., 1997; WO 97/41228).

Other screenable markers provide for visible light emission as a screenable phenotype. A screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell as fluorescence following illumination by particular wavelengths of light.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

D. Other Components

Sequences that are joined to the coding sequence of an expressed gene, which are removed post-translationally from the initial translation product and which facilitate the transport of the protein into or through intracellular or extracellular membranes, are termed transit or signal sequences. By facilitating the transport of the protein into compartments inside and outside the cell, these sequences may increase the accumulation of a gene product protecting the protein from intracellular proteolytic degradation. These sequences also allow for additional mRNA sequences from highly expressed genes to be attached to the coding sequence of the genes. Since mRNA being translated by ribosomes is more stable than naked mRNA, the presence of translatable mRNA 5′ of the gene of interest may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

In general embodiments of the invention, a nucleic acid segment encoding a leader peptide sequence upstream and in reading frame with a selected coding sequence is used in recombinant expression of the coding sequence in a host cell. In certain aspects, a leader peptide sequence comprises a signal recognized by a host cell that directs the transport of a polypeptide expressed in accordance with the invention through the outer membrane of a cell or into the periplasmic space. In aspects wherein the secreted product is transported into the extracellular medium, that product may be readily purified from host cells. In some aspects, the leader sequences may be removed by enzymatic cleavage. Such leader peptide sequences and nucleic acids encoding the sequences are known in the art.

Additionally, vectors may be constructed and employed in the intracellular targeting of a specific gene product within the cells of a transgenic organism or in directing a protein to the extracellular environment. This generally will be achieved by joining a DNA sequence encoding a transit or signal peptide sequence to the coding sequence of a particular gene. An intracellular targeting DNA sequence may be operably linked 5′ or 3′ to the coding sequence depending on the particular targeting sequence. The resultant transit, or signal, peptide will transport the protein to a particular intracellular, or extracellular destination, respectively, and will then be post-translationally removed.

It may also be desired that a transformation construct comprises a bacterial origin of replication. One example of such a origin of replication is a colE1 origin. It also may be desirable to include a bacterial selectable marker in the vector, for example, an ampicillin, tetracyclin, hygromycin, neomycin or chloramphenicol resistance gene (Bolivar et al., 1977). The Ap gene is an example of an E. coli marker gene which has been cloned and sequenced and which confers resistance to beta-lactam antibiotics such as ampicillin (nucleotides 4618 to 5478 of GenBank accession number U66885). Constructs comprising such elements may advantageously be propagated in bacterial cells such as E. coli cells.

E. Vector Construction

Expression constructs preferably comprise restriction endonuclease sites to facilitate vector construction. Particularly useful are unique restriction endonuclease recognition sites. Examples of such restriction sites include sites for the restriction endonucleases NotI, AatII, SacII and PmeI. Endonucleases preferentially break the internal phosphodiester bonds of polynucleotide chains. They may be relatively unspecific, cutting polynucleotide bonds regardless of the surrounding nucleotide sequence. However, the endonucleases which cleave only a specific nucleotide sequence are called restriction enzymes. Restriction endonucleases generally internally cleave DNA molecules at specific recognition sites, making breaks within “recognition” sequences that in many, but not all, cases exhibit two-fold symmetry around a given point. Such enzymes typically create double-stranded breaks.

Many of these enzymes make a staggered cleavage, yielding DNA fragments with protruding single-stranded 5′ or 3′ termini. Such ends are said to be “sticky” or “cohesive” because they will hydrogen bond to complementary 3′ or 5′ ends. As a result, the end of any DNA fragment produced by an enzyme, such as EcoRI, can anneal with any other fragment produced by that enzyme. This properly allows splicing of foreign genes into plasmids, for example. Some restriction endonucleases that may be particularly useful with the current invention include HindIII, PstI, EcoRI, and BamHI.

Some endonucleases create fragments that have blunt ends, that is, that lack any protruding single strands. An alternative way to create blunt ends is to use a restriction enzyme that leaves overhangs, but to fill in the overhangs with a polymerase, such as klenow, thereby resulting in blunt ends. When DNA has been cleaved with restriction enzymes that cut across both strands at the same position, blunt end ligation can be used to join the fragments directly together. The advantage of this technique is that any pair of ends may be joined together, irrespective of sequence.

Those nucleases that preferentially break off terminal nucleotides are referred to as exonucleases. For example, small deletions can be produced in any DNA molecule by treatment with an exonuclease which starts from each 3′ end of the DNA and chews away single strands in a 3′ to 5′ direction, creating a population of DNA molecules with single-stranded fragments at each end, some containing terminal nucleotides. Similarly, exonucleases that digest DNA from the 5′ end or enzymes that remove nucleotides from both strands have often been used. Some exonucleases which may be particularly useful in the present invention include Bal31, SI, and ExoIII. These nucleolytic reactions can be controlled by varying the time of incubation, the temperature, and the enzyme concentration needed to make deletions. Phosphatases and kinases also may be used to control which fragments have ends which can be joined. Examples of useful phosphatases include shrimp alkaline phosphatase and calf intestinal alkaline phosphatase. An example of a useful kinase is T4 polynucleotide kinase.

Once the source DNA sequences and vector sequences have been cleaved and modified to generate appropriate ends they are incubated together with enzymes capable of mediating the ligation of the two DNA molecules. Particularly useful enzymes for this purpose include T4 ligase, E. coli ligase, or other similar enzymes. The action of these enzymes results in the sealing of the linear DNA to produce a larger DNA molecule containing the desired fragment (see, for example, U.S. Pat. Nos. 4,237,224; 4,264,731; 4,273,875; 4,322,499 and 4,336,336, which are specifically incorporated herein by reference).

It is to be understood that the termini of the linearized plasmid and the termini of the DNA fragment being inserted must be complementary or blunt in order for the ligation reaction to be successful. Suitable complementarity can be achieved by choosing appropriate restriction endonucleases (i.e., if the fragment is produced by the same restriction endonuclease or one that generates the same overhang as that used to linearize the plasmid, then the termini of both molecules will be complementary). As discussed previously, in one embodiment of the invention, at least two classes of the vectors used in the present invention are adapted to receive the foreign oligonucleotide fragments in only one orientation. After joining the DNA segment to the vector, the resulting hybrid DNA can then be selected from among the large population of clones or libraries.

F. Utilization of Expression Constructs

Introduction of expression vectors into host cells in accordance with the invention will find use for the introduction of one or more new traits to the host cell. One example of such a trait is the ability to produce a heterologous protein. Potentially any of the many techniques known in the art for introducing the vector DNA may be employed, whereby the host becomes capable of efficient expression of the inserted sequences. Such expression can be obtained by operably linking a promoter, coding sequence and sequence containing transcription termination signals (a “terminator”). That is, the promoter effects proper expression of the protein or, if a signal sequence is present, the signal sequence-protein complex and the terminator effects proper termination of transcription and polyadenylation. In case a signal sequence is used, the signal sequence is linked in the proper reading frame to the protein gene in such a manner that the last codon of the signal sequence is directly linked to the first codon of the gene for the protein. The signal sequence, if present, has its own ATG for translation initiation.

II. Promoters for Modification of Plant Phenotypes

Modification of plant phenotypes requires use of promoters with the appropriate expression profile. Tissue-specific promoters may find use, for example, in modifying specific tissues within a plant while avoiding expression in other plant tissues, which may decrease plant productivity and/or present complicate regulatory hurdles. Alternatively, it may be desirable to use inducible promoters, for example, in the case of transgenes conferring resistance to environmental conditions such as osmotic stress, pest attack such as insect predation, or other environmental stresses such as nitrogen deprivation. In view of the importance of promoter elements having different expression profiles, the inventors carried out studies for the identification of promoters having a diverse group of expression profiles. Examples of some specific plant phenotypic modifications taken into consideration in identifying useful expression profiles are described below. The promoters provided by the invention may find use in such applications or any other desired uses.

One example of a modification that may be made to plants is to lignin content. Lignin is a major structural component of secondarily thickened plant cell walls. It is a complex polymer of hydroxylated and methoxylated phenylpropane units, linked via oxidative coupling (Boudet et al., 1995). Because of the negative effects of lignin on forage quality, there is considerable interest in genetic manipulation to alter the quantity and/or quality of the lignin polymer (Dixon et al., 1996). At the same time, lignin is important for stem rigidity and hydrophobicity of vascular elements, and, particularly in cereal crops, may be an important inducible defensive barrier against fungal pathogen attack (Beardmore et al., 1983). Thus, lignin modification must not compromise basic functions for the plant and thereby result in negative traits such as lodging or disease susceptibility. The key here is critical choice of both transgene and gene promoter. Potential improvements to forage quality associated with a reduction in lignin content, or changes in lignin quality, are summarized in Table 1.

TABLE 1
Potential benefits of transgenic alfalfa with improved cell wall digestibility

Increased energy from forage
Dietary fiber is required for rumen health; increased digestibility of this
fiber will result in more energy for milk/beef production.
Fiber digestibility will likely become a major limiting factor in further
increasing milk production in the U.S.
Increased milk/beef production potential
USDFRC estimates that a 10% increase in fiber digestibility would result
in an annual $350 million increase in milk/beef production.
Decreased generation of manure
USDFRC estimates that a 10% increase in fiber digestibility = 2.8 million
tons decrease in manure solids produced each year.

To date, attempts to genetically modify lignin in forage crops have targeted only three enzymes of the monolignol pathway, caffeic acid 3-O-methyltransferase (COMT), caffeoyl CoA 3-O-methyltransferase (CCoAOMT) and cinnamyl alcohol dehydrogenase (CAD). Constitutive cauliflower mosaic virus ³⁵S promoter-driven antisense reduction of COMT to less than 5% of wild-type values in the tropical pasture legume Stylosanthes humilis resulted in no apparent reduction in lignin levels but in a strong reduction in S lignin based on histochemical analysis (Rae et al., 2001). In vitro digestibility of stem material in rumen fluid was increased by up to 10% in the transgenic plants exhibiting strongest COMT down-regulation. Up to 30% decreases in Klason lignin levels were observed in transgenic alfalfa in which COMT down-regulation was targeted using the vascular-tissue specific bean PAL2 promoter, although acetyl bromide soluble lignin was not reduced. Use of this promoter resulted in near total down-regulation of COMT transcripts and protein (Guo et al., 2000) whereas earlier attempts at COMT down-regulation in alfalfa using the ³⁵S promoter were less effective, indicating the critical importance of promoter selection. COMT down-regulation in alfalfa was shown to lead to a loss of S residues in both the P-O-4-linked uncondensed fraction (the major fraction in most lignins) and in the condensed fraction resolved as a range of differently linked dimers (Guo et al., 2000). The effect of COMT down-regulation on S lignin therefore likely reflects a true metabolic reduction in S units, rather than a change in lignin composition resulting in appearance of more S units in the non-condensed fraction of the polymer. Loss of S-units was accompanied by appearance of 5-hydroxyguaiacyl residues in the lignin, and the presence of these residues, and their linkage to yield novel benzodioxane units, has been confirmed by the use of 2-dimensional nuclear magnetic resonance techniques (Marita et al., 2002). Thus, COMT down-regulation results in a striking alteration in lignin composition, and this has been confirmed in several different species, including both dicots and monocots (Jouanin et al., 2000; Ralph et al., 2001; Piquemal et al., 2002).

Analysis of in rumen digestibility of transgenic alfalfa in fistulated steers revealed that down-regulation of either COMT or CCoAOMT resulted in significant improvements in digestibility (Guo et al., 2001). Particularly striking was the observation that the digestion kinetics of forage from CCoAOMT down-regulated plants were biphasic, with digestion continuing beyond the time when it had ceased for forage from wild-type and COMT down-regulated plants. The OMT down-regulated lines have been crossed with an elite commercial alfalfa cultivar, and the improved digestibility trait has been shown to hold up in large scale field trials in Idaho, Wisconsin and Indiana. Currently, attempts are in progress to introduce the improved digestibility trait into a “Roundup-Ready” background for commercialization. Promoters with optimal expression profiles for such modification are therefore needed.

Condensed tannins (CTs, also known as proanthocyanidins) are phenolic polymers that bind to protein. They are synthesized by a branch of the flavonoid pathway. Although CTs occur in the fruits and seeds of many plants, they are either absent or present in very low amounts in many major forage sources such as alfalfa, white clover, corn silage, corn grain and soybean. The presence of CTs in the leaves of forage plants protects ruminant animals against pasture bloat and improves their nitrogen nutrition by increasing the amount of by-pass protein (dietary protein entering the small intestine from the rumen) (Broderick, 1995; Aerts et al., 1999; Barry and McNabb, 1999; Coulman et al., 2000; McMahon et al., 2000). In laboratory studies, binding of feed proteins with modest amounts of tannins (around 2-4% of dry matter) reduced both proteolysis during ensiling and rumen fermentation. In studies performed with sheep in New Zealand (Douglas et al., 1999), increasing dietary tannin from trace amounts to 4% of dry matter increased by-pass protein, and a diet containing only 2% tannin strongly increased absorption of essential amino acids by the small intestine by up to 60%. Milk production of non-supplemented Holstein cows is significantly increased by tannins in birdsfoot trefoil. These, and other advantages of the presence of tannins in forage crops, are listed in Table 2. At the same time, high concentrations of tannins can decrease palatability of forages, and can negatively impact nutritive value (Smulikowska et al., 2001).

The problem for engineering tannins into tissues of a plant that do not normally make them is that the biosynthetic pathways specific for the formation of CTs are still poorly understood. Most progress on tannin biosynthesis and its regulation has been made in non-forage species, by using genetic approaches in barley (which accumulates low molecular weight proanthocyanidin polymers lacking (−)-epicatechin, and in Arabidopsis thaliana, where mutants impaired in CT production can be readily scored by their transparent seed testa (Shirley et al., 1995).

TABLE 2
Potential benefits of transgenic forage crops with low
(2-4% dry weight) levels of condensed tannins.

Reduced rumen fermentation leading to a reduction
in incidence of pasture bloat
Reduction in methane gas emissions from ruminants
Reduced protein degradation during ensiling
Improved absorption of essential amino acids, leading to
increased meat, milk and wool production
Reduced excretion of soluble nitrogen in the urine
Reduced mineralization of carbon and nitrogen in the soil

Mutations in the BANYULS (BAN) gene (named after the color of a French red wine) result in precocious accumulation of red anthocyanins (flower pigments) and loss of CTs in the Arabidopsis seed coat (Devic et al., 1999). On this basis, and the amino acid sequence similarity of BAN to that of dihydroflavonol reductase, an enzyme that catalyzes an earlier step in the flavonoid pathway, it was suggested that BAN encodes leucoanthocyanidn reductase (LAR) (Devic et al., 1999), an enzyme proposed to convert flavan-3,4-diols to 2,3-trans-flavan-3-ols such as (+)-catechin (Stafford and Lester, 1984; Tanner and Kristiansen, 1993), a “starter unit” for CT condensation. However, it has recently been shown that BANYULS from both Arabidopsis and Medicago truncatula is a novel anthocyanidin reductase that converts anthocyanin to the corresponding 2,3-cis-flavan-3-ol such as (−)-epicatechin (Xie et al., 2003). The CT from Medicago seed coat consists of 4→8 linked (−)-epicatechin residues with a (+)-catechin residue as “starter” -(Koupai-Abyazani et al., 1993), a common structure among CTs. Thus, BAN activity may be involved in the biosynthesis of the repeating units in many CTs.

Introduction of Medicago BAN into transgenic tobacco under control of the ³⁵S promoter resulted in a depletion of the pink anthocyanin pigmentation in the flowers, and accumulation of material that stained with the tannin-specific reagents dimethylaminocinnamaldehyde and butanol-HCl (Xie et al., 2003). This material appears to be a polymeric condensed tannin based on its behavior on cellulose and Sephadex LH20 chromatography. Thus, it appears possible to produce CTs in tobacco flowers by simple ectopic expression of the BAN gene. Preliminary evidence indicates that tobacco flowers may naturally produce CTs, although at very low levels, and it has proven possible, by ectopic expression of transcription factors, to increase CT production in leaves of species that naturally accumulate these compounds (Robbins et al., 2003). However, formation of the two monomer types typical of alfalfa seed coat CT will require both BAN (for production of (−)-epicatechin) and a second enzyme for production of the (+)-catechin starter. This second enzyme might be a leucoanthocyanidin reductase (Tanner and Kristiansen, 1993), or perhaps a form of BAN that produces the flavan 3-ol with the 2,3-trans stereochemistry of (+)-catechin. In Arabidopsis, a number of transcription factors (Nesi et al., 2001), as well as a multidrug resistance type transporter (Debeaujon et al., 2001), are required for accumulation of CTs in the seed coat. It is likely that tissues that do not naturally accumulate CTs will, at minimum, require a source of anthocyanin, enzymes for formation of flavan-3-ols such as catechin and epicatechin, and transporter proteins to move the monomeric units in to the vacuole. Whether a specific enzyme is required for polymerization of the monomers has been debated for many years, but is yet to be resolved.

Production of a source of anthocyanin for CT synthesis in leaves is less of a problem than would at first sight appear. In fact, several forage legumes, such as white clover and barrel medic, contain an anthocyanin “spot” on the leaves, and the size of this spot appears to be under both genetic and environmental control. Furthermore, although anthocyanin biosynthesis requires many enzymes, it is possible to coordinately induce the pathway by ectopic expression of certain transcription factors such as the PAP-1 gene of Arabidopsis (Borevitz et al., 2001) or MYB and MYC genes from corn (Grotewold et al., 1998). Availability of transgenic plants accumulating the monomers necessary for CT biosynthesis will provide a basis for discovery of the downstream genes necessary for CT assembly. Although over 35 years of biochemical studies have failed to provide an answer as to how CT assembly is regulated, genomics/bioinformatic approaches can provide sets of candidate genes that can be readily tested by either stable or transient expression in a genetic background producing the monomers. Again, the key to success is a combination of the necessary transgenes under control of suitable developmentally controlled promoters.

Likely results of phenotypic engineering in agriculture will include bloat-safe alfalfa (Coulman et al., 2000), which will also significantly reduce greenhouse gas emission from cattle (J. Lee, AgResearch New Zealand, media release, May 2002), have improved silage quality (Albrecht and Muck, 1991), and increase the efficiency of alfalfa protein utilization by dairy cows, leading to reduced urine-N losses to the environment and a decreased requirement for feeding of supplemental protein (Broderick, 1995). CTs are also of considerable importance for human health and have been implicated in improving cardiovascular health and preventing urinary tract infections (Bagchi et al., 2000; Foo et al., 2000). They are also critical for flavor and astringency in wine, tea and other beverages.

Triterpene glycoside saponins are attracting increasing interest in view of their multiple biological activities (Table 3). These both positively and negatively impact plant traits, and can be divided into properties beneficial for plant protection, negatively impacting forage quality (Cheeke, 1976; Oleszek, 1996; Small, 1996; Oleszek et al., 1999), or of biomedical significance. Poultry are particularly sensitive to triterpene saponins, and this limits the use of alfalfa as a poultry feed. Alfalfa could otherwise be a feed of choice, because it results in eggs with a rich golden yolk. Despite the obvious interest in facilitating or inhibiting production of triterpene saponins for crop improvement or development of pharmacological agents, most of the steps in their biosynthesis remain uncharacterized at the molecular level.

In many plant species, the triterpene saponins form a relatively complex class of molecules. Those from alfalfa (and soybean) have been studied for many years (Pedersen et al., 1967; Oleszek, 1996). M. truncatula is an excellent model in which to understand the biosynthesis of triterpene saponins, making use of the extensive genomics resources for discovery of both biosynthetic and regulatory genes. The idea is that a better understanding of the biosynthetic pathways and their control points will facilitate engineering to alter the content of saponins such that forage quality will be improved but the defensive functions of saponins for the plant will be in large part maintained. Again, having recourse to promoters with tightly defined tissue- and inducer-specificity will be critical to this endeavor.

TABLE 3
Biological activities of triterpene saponins

Functions in plant defense
Allelopathic
Antifungal
Anti-insect
Properties impacting forage quality
Toxic to monogastrics
Anti-palatability
Reduce forage digestibility
Pharmacological/biomedical activities
Anti-cholesterol
Anti-cancer (eg. avicins from Acacia victoriae)
Adjuvant
Hemolytic

Lignin (primarily stem vascular specific), tannins (leaf specific) and triterpene saponins, which may need differential root and leaf specificity, are presented as just three examples of targeted traits for forage legume improvement needing diverse promoter elements. Others include aluminum tolerance (likely a root-specific process), disease and pest resistance (in response to fungi, bacteria, virus, nematodes, herbivores, aphids, etc), flower color and fragrance, among others. Clearly, the identification of gene promoters with strong functionality and specificity for legume systems is now a critical requirement for plant engineering and particularly legume and forage biotechnology. The potential uses of such promoters is outlined in Table 4.

TABLE 4
Promoter expression profiles and examples of use for
producing transgenic plants with improved characteristics

| Expression | |
|---|---|
| pattern of promoter | Examples of use |
| Constitutive | Genes of condensed tannin biosynthesis for |
| | bloat reduction; genes encoding proteins |
| | with increased nutritional value; genes that |
| | improve silage properties. |
| Root-specific | Genes for resistance to root pests or |
| | pathogens; genes that promote nodulation |
| | or mycorrhizal interactions; genes for |
| | phosphate or mineral nutrient uptake; genes |
| | for aluminum avoidance/detoxification. |
| Nodule-specific | Genes for improved nodulation and |
| | nitrogen fixation efficiency |
| Mycorrhizal-specific | Genes for improved phosphate acquisition |
| Phosphate-starvation | Genes encoding high affinity phosphate |
| inducible | transporters; genes for signal molecules to |
| | promote mycorrhizal colonization |
| Nitrogen-starvation | Genes leading to signals for establishment |
| inducible | of nodulation |
| Drought-inducible | Genes for drought tolerance |
| Stem specific | Genes for modification of lignin content |
| | and composition, lignin/polysaccharide |
| | cross-linking, or other factors affecting |
| | digestibility. Genes to improve stem |
| | rigidity (prevent lodging). |
| Developing seed specific | Genes for storage proteins, tannins, |
| | vaccines and other pharmaceutical proteins |
| UV-inducible | Genes for metabolic “sunscreens” |
| Fungal infection inducible | Genes for fungal resistance, particularly |
| | defenses that may be phytotoxic when |
| | expressed constitutively (eg. certain natural |
| | products) |
| Insect damage inducible | Genes encoding anti-herbivore proteins or |
| | pathways |

III. Methods for Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Tomes et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattachadjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al, 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

IV. Production and Characterization of Stably Transformed Plants

After effecting delivery of exogenous DNA to recipient cells, the next steps generally concern identifying the transformed cells and plants grown therefrom. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In one embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, can then be allowed to mature into plants. Developing plantlets may be transferred to soiless plant growth mix, and hardened, e.g., in an environmentally controlled chamber, for example, at about 85% relative humidity, 600 ppm CO₂, and 25-250 microeinsteins m⁻² S⁻¹ of light. Plants are preferably matured either in a growth chamber or greenhouse. Plants can be regenerated from about 6 wk to 10 months after a transformant is identified, depending on the initial tissue. During regeneration, cells may be grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

V. Site Specific Integration or Excision of Transgenes

In one embodiment of the invention, techniques for the site-specific integration or excision of transformation constructs may be used. An advantage of site-specific integration or excision is that it can be used to overcome problems associated with conventional transformation techniques, in which transformation constructs typically randomly integrate into a host genome in multiple copies. This random insertion of introduced DNA into the genome of host cells can be lethal if the foreign DNA inserts into an essential gene. In addition, the expression of a transgene may be influenced by “position effects” caused by the surrounding genomic DNA. Further, because of difficulties associated with cells possessing multiple transgene copies, including gene silencing, recombination and unpredictable inheritance, it is typically desirable to control the copy number of the inserted DNA, often only desiring the insertion of a single copy of the DNA sequence.

Site-specific integration or excision of transgenes or parts of transgenes can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. In plant cells, foreign DNA molecules find homologous sequences in the cell's genome and recombine at a frequency of approximately 0.5-4.2×10⁻⁴. Thus any transformed cell that contains an introduced DNA sequence integrated via homologous recombination will also likely contain numerous copies of randomly integrated introduced DNA sequences. Therefore, to maintain control over the copy number and the location of the inserted DNA, these randomly inserted DNA sequences can be removed. One manner of removing these random insertions is to utilize a site-specific recombinase system. In general, a site specific recombinase system consists of three elements: two pairs of DNA sequence (the site-specific recombination sequences) and a specific enzyme (the site-specific recombinase). The site-specific recombinase will catalyze a recombination reaction only between two site-specific recombination sequences.

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of CaMV (Golic and Lindquist, 1989), the Gin recombinase of phage Mu (Maeser et al., 1991), the Pin recombinase of E. coli (Enomoto et al., 1983), and the R/RS system of the pSR1 plasmid (Araki et al., 1992). The bacteriophage P1 Cre/10× and the CaMV FLP/FRT systems constitute two particularly useful systems for site specific integration or excision of transgenes. In these systems, a recombinase (Cre or FLP) will interact specifically with its respective site-specific recombination sequence (10× or FRT, respectively) to invert or excise the intervening sequences. The sequence for each of these two systems is relatively short (34 bp for 10× and 47 bp for FRT) and therefore, convenient for use with transformation vectors.

The FLP/FRT recombinase system has been demonstrated to function efficiently. Experiments on the performance of the FLP/FRT system indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible GAL1 promoter and this Cre gene was located on an autonomously replicating yeast vector.

VI. Breeding Plants of the Invention

In addition to direct transformation of a particular plant genotype with constructs prepared according to the current invention, transgenic plants may be made by crossing a plant having a selected DNA of the invention to a second plant lacking the construct. For example, a selected DNA can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:
(a) plant seeds of the first (starting line) and second (donor plant line that comprises a transgene of the invention) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

Backcrossing is herein defined as the process including the steps of:
(a) crossing a plant of a first genotype containing a desired gene, DNA sequence or element to a plant of a second genotype lacking said desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

VII. Definitions

About: When used with respect to the length of a nucleic acid sequence, means plus or minus ten base pairs.

Expression cassette: A transformation construct from which non-essential portions have been removed prior to introduction into a host genome by genetic transformation. Preferred expression cassettes will comprise all of the genetic elements necessary to direct the expression of a selected gene. Expression cassettes prepared in accordance with the instant invention will include a promoter of the invention.

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette therefrom) into a cell in which that exogenous DNA is incorporated into a chromosome or is capable of autonomous replication.

Heterologous coding sequence: Any coding sequence other than the native coding sequence. A coding sequence is any nucleic acid sequence capable of being transcribed into an mRNA.

Promoter: A recognition site on a DNA sequence or group of DNA sequences that provides an expression control element for a structural gene and to which RNA polymerase specifically binds and initiates RNA synthesis (transcription) of that gene.

Selected DNA: A DNA segment which one desires to introduce into a genome by genetic transformation.

Selected Gene: A gene which one desires to have expressed in a transgenic cell or organism comprising such a cell. A selected gene may be native or foreign to a host genome, but where the selected gene is present in the host genome, will typically include one or more regulatory or functional elements which differ from native copies of the gene.

Transformation construct: A chimeric DNA molecule which is designed for introduction into a host genome by genetic transformation. Preferred transformation constructs will comprise all of the genetic elements necessary to direct the expression of one or more exogenous genes. Transformation constructs prepared in accordance with the instant invention will include a promoter of the invention. The term “transformation construct” specifically includes expression cassettes.

Transgene: A segment of DNA which has been incorporated into a host genome or is capable of autonomous replication in a host cell and is capable of causing the expression of one or more cellular products. Exemplary transgenes will provide the host cell, or organisms comprising such a cell, with a novel phenotype relative to the corresponding non-transformed cell or organism. Transgenes may be directly introduced into a cell genetic transformation, or may be inherited from a cell of any previous generation which was transformed with the DNA segment.

Transgenic cell: A cell or a progeny cell of any generation derived therefrom, wherein the DNA of the cell or progeny thereof contains an introduced exogenous DNA segment not originally present in a non-transgenic cell of the same strain. The transgenic cell may additionally contain sequences which are native to the cell being transformed, but wherein the “exogenous” gene has been altered in order to alter the level or pattern of expression of the gene.

Vector: A DNA molecule capable of replication in a host cell and/or to which another DNA segment can be operatively linked so as to bring about replication of the attached segment.

### VIII. Examples

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Discovery of Tissue- and Inducer-Specific Gene Promoters from Medicago Truncatula

A data mining approach was developed for the identification and isolation for promoters having the following overall expression patterns:
Constitutive (expressed in all tissue types for which cDNA libraries had been sequenced)
Root specific (only expressed in libraries containing root tissue)
Root up-regulated (most strongly expressed in roots, but also expressed at low levels in other tissues)
Nodule specific (found in nodules and nodulated roots, but not in non-nodulated roots)
Nodule up-regulated (most strongly expressed in nodulated roots, but also expressed at low levels in other tissues)
Mycorrhizal specific (only found in mycorrhizal colonized roots)
Phosphate stress specific (only found in phosphate starved plants)
Phosphate starvation-inducible (most strongly expressed in phosphate-starved plants, but also expressed at low levels in the absence of phosphate starvation)
Nitrogen starvation-inducible (most strongly expressed in nitrogen-starved plants, but also expressed at low levels in the absence of phosphate starvation)
Drought specific (only expressed in drought-stressed seedlings)
Stem specific
Young developing seed specific
UV-inducible (only expressed following exposure of plants to high intensity UV light)
Fungal-infected leaf specific
Insect damaged leaf specific.

Large-scale EST sequencing has been used for rapid gene discovery in many plant species. Currently, the public EST data set of Medicago truncatula has been obtained from over 35 cDNA libraries representing different tissue types and/or various experimental treatments. These EST sequences have been assembled into contigs by The Institute for Genomic Research (TIGR) to generate a non-redundant data set, called M truncatula Gene Index or MtGI (Quackenbush et al. 2000). Furthermore, it is possible to roughly estimate the expression pattern of an MtGI gene by counting the frequency of its tags in different cDNA libraries (Ewing et al. 1999). This method, called EST counting or ‘electronic northern’, has been used to select some interesting targets for studies of rhizobial and arbuscular mycorrhizal symbioses in M. truncatula (Journet et al. 2002; Fedorova et al. 2002).

Data sets were obtained of MtGI version 5.0 (released on May 3, 2002) from TIGR through an academic/not-for-profit use license (www.tigr.org/tdb/tgi/). M. truncatula ESTs and cDNA library information were obtained from NCBI (http://www.ncbi.nlm.nih.gov/dbEST/). The data sets were then imported into a local database (MtGenes) to manage the data and compute the EST expression pattern for each MtGI gene. First, M. truncatula cDNA libraries constructed from similar plant sources and experimental conditions but from different laboratories were combined into a single set. For example, the “nodulated roots” set contains four cDNA libraries constructed from roots 1-4 days after nodulation. In this way, 21 different library sets were identified. Second, for each gene, its EST count in each of the library sets was computed. The counts were further normalized by the set size (number of ESTs). Thus, relative gene expression levels were represented by the frequency of tag occurrence. Third, genes with EST expression most highly specific to or up-regulated in a particular library set were selected as the initial targets. The constitutive gene targets were almost uniformly expressed in all the library sets. Overall, over 300 targets were selected for different expression patterns.

To identify promoters for these targets, the emerging M. truncatula genomic sequences were searched using the BLAST program (ftp://ftp.ncbi.nih.gov/blast/executables/). The genomic sequence data set was downloaded from GenBank. To the end, about 30 genomic contigs showed over 95% identity with the targets in the aligned regions. Gene structures of these genomic sequences, including putative intron/exon boundaries and transcription start sites, were then predicted using the FGeneSH software tool (Salamov and Solovyev 2000). The predicted coding sequences (CDS) were further aligned with the MtGI sequences to validate the genomic target authenticity and prediction accuracy. In this way, promoter regions were identified for 26 M. truncatula genes with defined tissue-/inducer-specificity. The DNA sequences of these upstream regions are shown in SEQ ID NOs:1-26. The expression specificities were confirmed by the EST counts reported in Tables 5-6.

TABLE 5
Promoter sequences identified and associated expression profiles

| PROMOTER | |
|---|---|
| SEQUENCE ID NO | EXPRESSION PROFILE |
| SEQ ID NO: 1 | constitutive promoter sequence |
| SEQ ID NO: 2 | phosphate starvation specific promoter |
| | sequence |
| SEQ ID NO: 3 | nitrogen starvation upregulated promoter |
| | sequence |
| SEQ ID NO: 4 | drought upregulated promoter sequence |
| SEQ ID NO: 5 | mycorrhizal root specific promoter |
| | sequence |
| SEQ ID NO: 6 | nodulation specific promoter sequence |
| SEQ ID NO: 7 | nodulation specific promoter sequence |
| SEQ ID NO: 8 | drought specific promoter sequence |
| SEQ ID NO: 9 | insect herbivory leaf specific promoter |
| | sequence |
| SEQ ID NO: 10 | developing stem specific promoter |
| | sequence |
| SEQ ID NO: 11 | developing seed specific promoter |
| | sequence |
| SEQ ID NO: 12 | phosphate starvation upregulated promoter |
| | sequence |
| SEQ ID NO: 13 | constitutive promoter sequence |
| SEQ ID NO: 14 | UV irradiation induced promoter sequence |
| SEQ ID NO: 15 | constitutive promoter sequence |
| SEQ ID NO: 16 | developing seed specific promoter |
| | sequence |
| SEQ ID NO: 17 | nodulation upregulated promoter sequence |
| SEQ ID NO: 18 | fungus-infected leaf specific promoter |
| | sequence |
| SEQ ID NO: 19 | phosphate starvation specific promoter |
| | sequence |
| SEQ ID NO: 20 | developing flower specific promoter |
| | sequence |
| SEQ ID NO: 21 | root specific promoter sequence |
| SEQ ID NO: 22 | drought specific promoter sequence |
| SEQ ID NO: 23 | constitutive promoter sequence |
| SEQ ID NO: 24 | root upregulated promoter sequence |
| SEQ ID NO: 25 | root specific promoter sequence |
| SEQ ID NO: 26 | developing pod upregulated promoter |
| | sequence |

TABLE 6A

| | | | | Datasets | | | | |
|---|---|---|---|---|---|---|---|---|
| | | | | symbiosis | | | | root, |
| | | | | Dataset size | root, no | root, early | root, late | nodulation |
| | | | | Frequency (10-3) | nodulation | nodulation | nodulation | mixed |
| GB ID | TC # | Category | Annotation | Overall | 6705.0000 | 18292.0000 | 9694.0000 | 3299.0000 |
| AC126009 | TC43181 | Constitutive | Similar to 5- | 3.5815 | 4.3251 | 3.0614 | 3.7136 | 0.9094 |
| | | | methyltetrahydropteroyltriglutamate-- | | | | | |
| | | | homocysteine S-methyltransferase | | | | | |
| | | | [Arabidopsis thaliana] | | | | | |
| AC135505 | TC43151 | Constitutive | Similar to glyceraldehyde 3-phosphate | 2.1838 | 1.4914 | 2.2414 | 4.7452 | 1.5156 |
| | | | dehydrogenase, cytosolic (EC 1.2.1.12) | | | | | |
| | | | {Pisum sativum} | | | | | |
| AC122727 | TC51103 | Constitutive | Similar to FRUCTOSE- | 1.1531 | 1.0440 | 1.8587 | 2.2694 | 1.5156 |
| | | | BISPHOSPHATE ALDOLASE, | | | | | |
| | | | CYTOPLASMIC ISOZYME 2 | | | | | |
| AC126014 | TC51310 | Constitutive | Similar to L3 Ribosomal protein | 0.9143 | 1.3423 | 0.8200 | 0.6189 | 1.8187 |
| | | | {Medicago sativa} | | | | | |
| AC123575 | TC43967 | Root-specific | Peroxidase | 0.1281 | 0.4474 | 0.3280 | 0.0000 | 0.0000 |
| AC122162 | TC52517 | Root-specific | Putative CCCH-type zinc finger protein | 0.0641 | 0.1491 | 0.2733 | 0.3095 | 0.0000 |
| AC121240 | TC42993 | Root- | Putative senescence-associated | 0.5474 | 3.4303 | 1.7494 | 0.6189 | 0.6062 |
| | | upregulated | monooxygenase | | | | | |
| AC124953 | TC51588 | Nodulation- | Leghemoglobin | 0.1689 | 0.0000 | 0.0000 | 2.8884 | 0.3031 |
| | | specific | | | | | | |
| AC124953 | TC51080 | Nodulation- | Similar to leghemoglobin I - alfalfa | 0.1223 | 0.0000 | 0.0000 | 2.1663 | 0.0000 |
| | | specific | | | | | | |
| AC124963 | TC53871 | Nodulation- | Unknown protein | 0.0408 | 0.1491 | 0.2187 | 0.0000 | 0.0000 |
| | | upregulated | | | | | | |
| AC126787 | TC53652 | Mycorrhizal | Similar to glutathione transferase (EC | 0.0349 | 0.0000 | 0.0000 | 0.0000 | 0.0000 |
| | | root-specific | 2.5.1.18), 2,4-D inducible - soybean | | | | | |
| AC135396 | TC57723 | P starvation- | Unknown protein | 0.0116 | 0.0000 | 0.0000 | 0.0000 | 0.0000 |
| | | specific | | | | | | |
| AC130799 | TC56603 | P starvation- | Acetylglutamate kinase-like protein | 0.0175 | 0.0000 | 0.0000 | 0.0000 | 0.0000 |
| | | specific | | | | | | |
| AC126018 | TC52900 | P starvation- | Similar to PSI P700 apoprotein A2 | 0.0641 | 0.0000 | 0.0000 | 0.0000 | 0.0000 |
| | | upregulated | [Lotus japonicus] | | | | | |
| AC135797 | TC44600 | N starvation- | Putative clathrin assembly protein | 0.0524 | 0.0000 | 0.0547 | 0.0000 | 0.3031 |
| | | upregulated | | | | | | |
| AC126794 | TC50291 | Drought- | Similar to GCN4-complementing | 0.0116 | 0.0000 | 0.0000 | 0.0000 | 0.0000 |
| | | specific | protein homolog [Arabidopsis thaliana] | | | | | |
| AC135605 | TC50939 | Drought- | Similar to hypothetical protein | 0.0116 | 0.0000 | 0.0000 | 0.0000 | 0.0000 |
| | | specific | [Arabidopsis thaliana] | | | | | |
| AC126787 | TC45266 | Drought- | Unknown protein (Myb domain) | 0.0291 | 0.0000 | 0.0000 | 0.0000 | 0.0000 |
| | | upregulated | | | | | | |
| AC135467 | TC45846 | Dev stem- | Similar to laccase [Liriodendron | 0.0408 | 0.0000 | 0.0000 | 0.0000 | 0.0000 |
| | | specific | tulipifera] | | | | | |
| AC121233 | TC58764 | Dev flower- | Similar to putative protein [Arabidopsis | 0.0116 | 0.0000 | 0.0000 | 0.0000 | 0.0000 |
| | | specific | thaliana] | | | | | |
| AC135317 | TC43932 | Dev seed- | Late embryogenesis abundant (LEA) | 0.0582 | 0.0000 | 0.0000 | 0.0000 | 0.0000 |
| | | specific | protein | | | | | |
| AC127429 | TC53381 | Dev seed- | Similar to dehydration-induced protein | 0.0466 | 0.0000 | 0.0000 | 0.0000 | 0.0000 |
| | | specific | RD22-like protein [Gossypium | | | | | |
| | | | hirsutum] | | | | | |
| AC130800 | TC52422 | Dev Pod- | Putative cytochrome P450 | 0.0699 | 0.0000 | 0.0547 | 0.0000 | 0.0000 |
| | | upregulated | | | | | | |
| AC126012 | TC48419 | UV | WD40 protein | 0.0175 | 0.0000 | 0.0000 | 0.0000 | 0.0000 |
| | | irradiation | | | | | | |
| AC124958 | TC56930 | Fungus- | Unknown protein | 0.0116 | 0.0000 | 0.0000 | 0.0000 | 0.0000 |
| | | infected leaf | | | | | | |
| AC135162 | TC48368 | Insect | Similar to cytochrome P450 [Pyrus | 0.0175 | 0.0000 | 0.0000 | 0.0000 | 0.0000 |
| | | herbivory | communis] | | | | | |
| | | leaf | | | | | | |
| AC126782* | TC48366 | Insect | Similar to putative protein [Arabidopsis | 0.0175 | 0.0000 | 0.0000 | 0.0000 | 0.0000 |
| | | herbivory | thaliana] | | | | | |
| | | leaf | | | | | | |

*No promoter sequence available.

TABLE 6B

| | | | | | | root, | | |
|---|---|---|---|---|---|---|---|---|
| | | | | root, nitrogen- | root, | phosphate- | root, | flower, |
| | | | | starved | mycorrhizal | starved | elicited | developing |
| GB ID | TC # | Category | Annotation | 7939.0000 | 15969.0000 | 4625.0000 | 8011.0000 | 6724.0000 |
| AC126009 | TC43181 | Constitutive | Similar to 5- | 13.0999 | 2.2544 | 0.0000 | 0.8738 | 3.8667 |
| | | | methyltetrahydropteroyltriglutamate-- | | | | | |
| | | | homocysteine S-methyltransferase | | | | | |
| | | | [Arabidopsis thaliana] | | | | | |
| AC135505 | TC43151 | Constitutive | Similar to glyceraldehyde | 3.4009 | 2.0039 | 1.2973 | 1.3731 | 2.3795 |
| | | | 3-phosphate dehydrogenase, | | | | | |
| | | | cytosolic (EC 1.2.1.12) | | | | | |
| | | | {Pisum sativum} | | | | | |
| AC122727 | TC51103 | Constitutive | Similar to FRUCTOSE- | 3.1490 | 0.7515 | 0.4324 | 1.7476 | 1.0410 |
| | | | BISPHOSPHATE ALDOLASE, | | | | | |
| | | | CYTOPLASMIC ISOZYME 2 | | | | | |
| AC126014 | TC51310 | Constitutive | Similar to L3 Ribosomal protein | 1.0077 | 0.5636 | 1.5135 | 1.1235 | 1.7847 |
| | | | {Medicago sativa} | | | | | |
| AC123575 | TC43967 | Root-specific | Peroxidase | 1.1336 | 0.0000 | 0.6486 | 0.0000 | 0.0000 |
| AC122162 | TC52517 | Root-specific | Putative CCCH-type zinc finger | 0.0000 | 0.0000 | 0.0000 | 0.1248 | 0.0000 |
| | | | protein | | | | | |
| AC121240 | TC42993 | Root- | Putative senescence-associated | 0.7558 | 0.1879 | 0.0000 | 0.4993 | 0.2974 |
| | | upregulated | monooxygenase | | | | | |
| AC124953 | TC51588 | Nodulation- | Leghemoglobin | 0.0000 | 0.0000 | 0.0000 | 0.0000 | 0.0000 |
| | | specific | | | | | | |
| AC124953 | TC51080 | Nodulation- | Similar to leghemoglobin I - alfalfa | 0.0000 | 0.0000 | 0.0000 | 0.0000 | 0.0000 |
| | | specific | | | | | | |
| AC124963 | TC53871 | Nodulation- | Unknown protein | 0.0000 | 0.0000 | 0.0000 | 0.1248 | 0.0000 |
| | | upregulated | | | | | | |
| AC126787 | TC53652 | Mycorrhizal | Similar to glutathione transferase (EC | 0.0000 | 0.3757 | 0.0000 | 0.0000 | 0.0000 |
| | | root-specific | 2.5.1.18), 2,4-D inducible - soybean | | | | | |
| AC135396 | TC57723 | P starvation- | Unknown protein | 0.0000 | 0.0000 | 0.2162 | 0.0000 | 0.0000 |
| | | specific | | | | | | |
| AC130799 | TC56603 | P starvation- | Acetylglutamate kinase-like protein | 0.0000 | 0.0000 | 0.0000 | 0.0000 | 0.0000 |
| | | specific | | | | | | |
| AC126018 | TC52900 | P starvation- | Similar to PSI P700 apoprotein A2 | 0.0000 | 0.0000 | 0.0000 | 0.0000 | 0.1487 |
| | | upregulated | [Lotus japonicus] | | | | | |
| AC135797 | TC44600 | N starvation- | Putative clathrin assembly protein | 0.5038 | 0.0626 | 0.0000 | 0.0000 | 0.1487 |
| | | upregulated | | | | | | |
| AC126794 | TC50291 | Drought- | Similar to GCN4-complementing | 0.0000 | 0.0000 | 0.0000 | 0.0000 | 0.0000 |
| | | specific | protein homolog [Arabidopsis | | | | | |
| | | | thaliana] | | | | | |
| AC135605 | TC50939 | Drought- | Similar to hypothetical protein | 0.0000 | 0.0000 | 0.0000 | 0.0000 | 0.0000 |
| | | specific | [Arabidopsis thaliana] | | | | | |
| AC126787 | TC45266 | Drought- | Unknown protein (Myb domain) | 0.0000 | 0.0000 | 0.0000 | 0.0000 | 0.1487 |
| | | upregulated | | | | | | |
| AC135467 | TC45846 | Dev stem- | Similar to laccase [Liriodendron | 0.0000 | 0.0000 | 0.0000 | 0.0000 | 0.0000 |
| | | specific | tulipifera] | | | | | |
| AC121233 | TC58764 | Dev flower- | Similar to putative protein | 0.0000 | 0.0000 | 0.0000 | 0.0000 | 0.2974 |
| | | specific | [Arabidopsis thaliana] | | | | | |
| AC135317 | TC43932 | Dev seed- | Late embryogenesis abundant (LEA) | 0.0000 | 0.0000 | 0.0000 | 0.0000 | 0.0000 |
| | | specific | protein | | | | | |
| AC127429 | TC53381 | Dev seed- | Similar to dehydration-induced | 0.0000 | 0.0000 | 0.0000 | 0.0000 | 0.0000 |
| | | specific | protein RD22-like protein | | | | | |
| | | | [Gossypium hirsutum] | | | | | |
| AC130800 | TC52422 | Dev Pod- | Putative cytochrome P450 | 0.0000 | 0.0000 | 0.0000 | 0.0000 | 0.0000 |
| | | upregulated | | | | | | |
| AC126012 | TC48419 | UV irradiation | WD40 protein | 0.0000 | 0.0000 | 0.0000 | 0.0000 | 0.0000 |
| AC124958 | TC56930 | Fungus- | Unknown protein | 0.0000 | 0.0000 | 0.0000 | 0.0000 | 0.0000 |
| | | infected leaf | | | | | | |
| AC135162 | TC48368 | Insect | Similar to cytochrome P450 [Pyrus | 0.0000 | 0.0000 | 0.0000 | 0.0000 | 0.0000 |
| | | herbivory leaf | communis] | | | | | |
| AC126782* | TC48366 | Insect | Similar to putative protein | 0.0000 | 0.0000 | 0.0000 | 0.0000 | 0.0000 |
| | | herbivory leaf | [Arabidopsis thaliana] | | | | | |

*No promoter sequence available.

TABLE 6C

| | | | | | | | | leaf, |
|---|---|---|---|---|---|---|---|---|
| | | | | seed, | pod, | seed, | leaf, | phosphate- |
| | | | | developing | developing | germinating | developing | starved |
| GB ID | TC # | Category | Annotation | 5616.0000 | 1915.0000 | 1524.0000 | 9415.0000 | 10188.0000 |
| AC126009 | TC43181 | Constitutive | Similar to 5- | 0.8903 | 14.6214 | 2.6247 | 1.9118 | 0.7852 |
| | | | methyltetrahydropteroyltriglutamate-- | | | | | |
| | | | homocysteine S-methyltransferase | | | | | |
| | | | [Arabidopsis thaliana] | | | | | |
| AC135505 | TC43151 | Constitutive | Similar to glyceraldehyde 3- | 0.7123 | 2.0888 | 0.0000 | 2.1243 | 3.0428 |
| | | | phosphate dehydrogenase, cytosolic | | | | | |
| | | | (EC 1.2.1.12) {Pisum sativum} | | | | | |
| AC122727 | TC51103 | Constitutive | Similar to FRUCTOSE- | 0.5342 | 2.6110 | 0.0000 | 0.2124 | 1.1779 |
| | | | BISPHOSPHATE ALDOLASE, | | | | | |
| | | | CYTOPLASMIC ISOZYME 2 | | | | | |
| AC126014 | TC51310 | Constitutive | Similar to L3 Ribosomal protein | 2.6709 | 0.0000 | 0.0000 | 0.6373 | 0.7852 |
| | | | {Medicago sativa} | | | | | |
| AC123575 | TC43967 | Root-specific | Peroxidase | 0.0000 | 0.0000 | 0.0000 | 0.0000 | 0.0000 |
| AC122162 | TC52517 | Root-specific | Putative CCCH-type zinc finger | 0.0000 | 0.0000 | 0.0000 | 0.0000 | 0.0000 |
| | | | protein | | | | | |
| AC121240 | TC42993 | Root- | Putative senescence-associated | 0.0000 | 0.0000 | 0.0000 | 0.4249 | 0.4908 |
| | | upregulated | monooxygenase | | | | | |
| AC124953 | TC51588 | Nodulation- | Leghemoglobin | 0.0000 | 0.0000 | 0.0000 | 0.0000 | 0.0000 |
| | | specific | | | | | | |
| AC124953 | TC51080 | Nodulation- | Similar to leghemoglobin I - alfalfa | 0.0000 | 0.0000 | 0.0000 | 0.0000 | 0.0000 |
| | | specific | | | | | | |
| AC124963 | TC53871 | Nodulation- | Unknown protein | 0.1781 | 0.0000 | 0.0000 | 0.0000 | 0.0000 |
| | | upregulated | | | | | | |
| AC126787 | TC53652 | Mycorrhizal | Similar to glutathione transferase | 0.0000 | 0.0000 | 0.0000 | 0.0000 | 0.0000 |
| | | root-specific | (EC 2.5.1.18), 2,4-D inducible - | | | | | |
| | | | soybean | | | | | |
| AC135396 | TC57723 | P starvation- | Unknown protein | 0.0000 | 0.0000 | 0.0000 | 0.0000 | 0.0982 |
| | | specific | | | | | | |
| AC130799 | TC56603 | P starvation- | Acetylglutamate kinase-like protein | 0.0000 | 0.0000 | 0.0000 | 0.0000 | 0.2945 |
| | | specific | | | | | | |
| AC126018 | TC52900 | P starvation- | Similar to PSI P700 apoprotein A2 | 0.1781 | 0.0000 | 0.0000 | 0.0000 | 0.5889 |
| | | upregulated | [Lotus japonicus] | | | | | |
| AC135797 | TC44600 | N starvation- | Putative clathrin assembly protein | 0.0000 | 0.0000 | 0.0000 | 0.0000 | 0.0982 |
| | | upregulated | | | | | | |
| AC126794 | TC50291 | Drought- | Similar to GCN4-complementing | 0.0000 | 0.0000 | 0.0000 | 0.0000 | 0.0000 |
| | | specific | protein homolog [Arabidopsis | | | | | |
| | | | thaliana] | | | | | |
| AC135605 | TC50939 | Drought- | Similar to hypothetical protein | 0.0000 | 0.0000 | 0.0000 | 0.0000 | 0.0000 |
| | | specific | [Arabidopsis thaliana] | | | | | |
| AC126787 | TC45266 | Drought- | Unknown protein (Myb domain) | 0.0000 | 0.0000 | 0.0000 | 0.1062 | 0.0000 |
| | | upregulated | | | | | | |
| AC135467 | TC45846 | Dev stem- | Similar to laccase [Liriodendron | 0.0000 | 0.0000 | 0.0000 | 0.0000 | 0.0000 |
| | | specific | tulipifera] | | | | | |
| AC121233 | TC58764 | Dev flower- | Similar to putative protein | 0.0000 | 0.0000 | 0.0000 | 0.0000 | 0.0000 |
| | | specific | [Arabidopsis thaliana] | | | | | |
| AC135317 | TC43932 | Dev seed- | Late embryogenesis abundant (LEA) | 1.7806 | 0.0000 | 0.0000 | 0.0000 | 0.0000 |
| | | specific | protein | | | | | |
| AC127429 | TC53381 | Dev seed- | Similar to dehydration-induced | 1.4245 | 0.0000 | 0.0000 | 0.0000 | 0.0000 |
| | | specific | protein RD22-like protein | | | | | |
| | | | [Gossypium hirsutum] | | | | | |
| AC130800 | TC52422 | Dev Pod- | Putative cytochrome P450 | 0.0000 | 3.6554 | 0.0000 | 0.0000 | 0.0982 |
| | | upregulated | | | | | | |
| AC126012 | TC48419 | UV | WD40 protein | 0.0000 | 0.0000 | 0.0000 | 0.0000 | 0.0000 |
| | | irradiation | | | | | | |
| AC124958 | TC56930 | Fungus- | Unknown protein | 0.0000 | 0.0000 | 0.0000 | 0.0000 | 0.0000 |
| | | infected leaf | | | | | | |
| AC135162 | TC48368 | Insect | Similar to cytochrome P450 [Pyrus | 0.0000 | 0.0000 | 0.0000 | 0.0000 | 0.0000 |
| | | herbivory | communis] | | | | | |
| | | leaf | | | | | | |
| AC126782* | TC48366 | Insect | Similar to putative protein | 0.0000 | 0.0000 | 0.0000 | 0.0000 | 0.0000 |
| | | herbivory | [Arabidopsis thaliana] | | | | | |
| | | leaf | | | | | | |

*No promoter sequence available.

TABLE 6D

| | | | | | leaf, | | | |
|---|---|---|---|---|---|---|---|---|
| | | | | leaf, insect | fungus- | cotyledon and | stem, | seedling, |
| | | | | herbivory | infected | leaf | developing | drought |
| GB ID | TC # | Category | Annotation | 10309.0000 | 9284.0000 | 2143.0000 | 10783.0000 | 9520.0000 |
| AC126009 | TC43181 | Constitutive | Similar to 5- | 4.3651 | 0.9694 | 1.3999 | 12.3342 | 3.8866 |
| | | | methyltetrahydropteroyltriglutamate-- | | | | | |
| | | | homocysteine S-methyltransferase | | | | | |
| | | | [Arabidopsis thaliana] | | | | | |
| AC135505 | TC43151 | Constitutive | Similar to glyceraldehyde 3- | 3.2981 | 1.8311 | 0.9333 | 1.7620 | 2.6261 |
| | | | phosphate dehydrogenase, cytosolic | | | | | |
| | | | (EC 1.2.1.12) {Pisum sativum} | | | | | |
| AC122727 | TC51103 | Constitutive | Similar to FRUCTOSE- | 0.3880 | 0.5386 | 0.4666 | 1.2983 | 0.7353 |
| | | | BISPHOSPHATE ALDOLASE, | | | | | |
| | | | CYTOPLASMIC ISOZYME 2 | | | | | |
| AC126014 | TC51310 | Constitutive | Similar to L3 Ribosomal protein | 0.2910 | 0.5386 | 0.0000 | 0.4637 | 1.0504 |
| | | | {Medicago sativa} | | | | | |
| AC123575 | TC43967 | Root-specific | Peroxidase | 0.0000 | 0.0000 | 0.0000 | 0.0000 | 0.0000 |
| AC122162 | TC52517 | Root-specific | Putative CCCH-type zinc finger | 0.0000 | 0.0000 | 0.0000 | 0.0000 | 0.1050 |
| | | | protein | | | | | |
| AC121240 | TC42993 | Root- | Putative senescence-associated | 0.1940 | 0.0000 | 0.0000 | 0.1855 | 0.2101 |
| | | upregulated | monooxygenase | | | | | |
| AC124953 | TC51588 | Nodulation- | Leghemoglobin | 0.0000 | 0.0000 | 0.0000 | 0.0000 | 0.0000 |
| | | specific | | | | | | |
| AC124953 | TC51080 | Nodulation- | Similar to leghemoglobin I - alfalfa | 0.0000 | 0.0000 | 0.0000 | 0.0000 | 0.0000 |
| | | specific | | | | | | |
| AC124963 | TC53871 | Nodulation- | Unknown protein | 0.0000 | 0.0000 | 0.0000 | 0.0000 | 0.0000 |
| | | upregulated | | | | | | |
| AC126787 | TC53652 | Mycorrhizal | Similar to glutathione transferase | 0.0000 | 0.0000 | 0.0000 | 0.0000 | 0.0000 |
| | | root-specific | (EC 2.5.1.18), 2,4-D inducible - | | | | | |
| | | | soybean | | | | | |
| AC135396 | TC57723 | P starvation- | Unknown protein | 0.0000 | 0.0000 | 0.0000 | 0.0000 | 0.0000 |
| | | specific | | | | | | |
| AC130799 | TC56603 | P starvation- | Acetylglutamate kinase-like protein | 0.0000 | 0.0000 | 0.0000 | 0.0000 | 0.0000 |
| | | specific | | | | | | |
| AC126018 | TC52900 | P starvation- | Similar to PSI P700 apoprotein A2 | 0.1940 | 0.1077 | 0.0000 | 0.0000 | 0.0000 |
| | | upregulated | [Lotus japonicus] | | | | | |
| AC135797 | TC44600 | N starvation- | Putative clathrin assembly protein | 0.0000 | 0.0000 | 0.0000 | 0.0000 | 0.0000 |
| | | upregulated | | | | | | |
| AC126794 | TC50291 | Drought- | Similar to GCN4-complementing | 0.0000 | 0.0000 | 0.0000 | 0.0000 | 0.2101 |
| | | specific | protein homolog [Arabidopsis | | | | | |
| | | | thaliana] | | | | | |
| AC135605 | TC50939 | Drought- | Similar to hypothetical protein | 0.0000 | 0.0000 | 0.0000 | 0.0000 | 0.2101 |
| | | specific | [Arabidopsis thaliana] | | | | | |
| AC126787 | TC45266 | Drought- | Unknown protein (Myb domain) | 0.0000 | 0.0000 | 0.0000 | 0.0000 | 0.2101 |
| | | upregulated | | | | | | |
| AC135467 | TC45846 | Dev stem- | Similar to laccase [Liriodendron | 0.0000 | 0.0000 | 0.0000 | 0.6492 | 0.0000 |
| | | specific | tulipifera] | | | | | |
| AC121233 | TC58764 | Dev flower- | Similar to putative protein | 0.0000 | 0.0000 | 0.0000 | 0.0000 | 0.0000 |
| | | specific | [Arabidopsis thaliana] | | | | | |
| AC135317 | TC43932 | Dev seed- | Late embryogenesis abundant (LEA) | 0.0000 | 0.0000 | 0.0000 | 0.0000 | 0.0000 |
| | | specific | protein | | | | | |
| AC127429 | TC53381 | Dev seed- | Similar to dehydration-induced | 0.0000 | 0.0000 | 0.0000 | 0.0000 | 0.0000 |
| | | specific | protein RD22-like protein | | | | | |
| | | | [Gossypium hirsutum] | | | | | |
| AC130800 | TC52422 | Dev Pod- | Putative cytochrome P450 | 0.1940 | 0.0000 | 0.0000 | 0.0000 | 0.0000 |
| | | upregulated | | | | | | |
| AC126012 | TC48419 | UV irradiation | WD4O protein | 0.0000 | 0.0000 | 0.0000 | 0.0000 | 0.0000 |
| AC124958 | TC56930 | Fungus- | Unknown protein | 0.0000 | 0.2154 | 0.0000 | 0.0000 | 0.0000 |
| | | infected leaf | | | | | | |
| AC135162 | TC48368 | Insect | Similar to cytochrome P450 [Pyrus | 0.2910 | 0.0000 | 0.0000 | 0.0000 | 0.0000 |
| | | herbivory leaf | communis] | | | | | |
| AC126782* | TC48366 | Insect | Similar to putative protein | 0.2910 | 0.0000 | 0.0000 | 0.0000 | 0.0000 |
| | | herbivory leaf | [Arabidopsis thaliana] | | | | | |

*No promoter sequence available.

TABLE 6E

| | | | | seedling, | cell culture, | root, nematode- |
|---|---|---|---|---|---|---|
| | | | | irradiated | elicited | infected |
| GB ID | TC # | Category | Annotation | 6748.0000 | 9859.0000 | 3154.0000 |
| AC126009 | TC43181 | Constitutive | Similar to 5- | 1.7783 | 0.8114 | 2.5365 |
| | | | methyltetrahydropteroyltriglutamate-- | | | |
| | | | homocysteine S-methyltransferase | | | |
| | | | [Arabidopsis thaliana] | | | |
| AC135505 | TC43151 | Constitutive | Similar to glyceraldehyde 3-phosphate | 2.0747 | 0.9129 | 0.6341 |
| | | | dehydrogenase, cytosolic (EC 1.2.1.12) | | | |
| | | | {Pisum sativum} | | | |
| AC122727 | TC51103 | Constitutive | Similar to FRUCTOSE-BISPHOSPHATE | 0.5928 | 0.6086 | 2.2194 |
| | | | ALDOLASE, CYTOPLASMIC ISOZYME 2 | | | |
| AC126014 | TC51310 | Constitutive | Similar to L3 Ribosomal protein {Medicago | 1.7783 | 0.9129 | 0.9512 |
| | | | sativa} | | | |
| AC123575 | TC43967 | Root-specific | Peroxidase | 0.0000 | 0.0000 | 0.3171 |
| AC122162 | TC52517 | Root-specific | Putative CCCH-type zinc finger protein | 0.0000 | 0.0000 | 0.0000 |
| AC121240 | TC42993 | Root- | Putative senescence-associated | 0.0000 | 0.1014 | 0.0000 |
| | | upregulated | monooxygenase | | | |
| AC124953 | TC51588 | Nodulation- | Leghemoglobin | 0.0000 | 0.0000 | 0.0000 |
| | | specific | | | | |
| AC124953 | TC51080 | Nodulation- | Similar to leghemoglobin I - alfalfa | 0.0000 | 0.0000 | 0.0000 |
| | | specific | | | | |
| AC124963 | TC53871 | Nodulation- | Unknown protein | 0.0000 | 0.0000 | 0.0000 |
| | | upregulated | | | | |
| AC126787 | TC53652 | Mycorrhizal | Similar to glutathione transferase (EC | 0.0000 | 0.0000 | 0.0000 |
| | | root-specific | 2.5.1.18), 2,4-D inducible - soybean | | | |
| AC135396 | TC57723 | P starvation- | Unknown protein | 0.0000 | 0.0000 | 0.0000 |
| | | specific | | | | |
| AC130799 | TC56603 | P starvation- | Acetylglutamate kinase-like protein | 0.0000 | 0.0000 | 0.0000 |
| | | specific | | | | |
| AC126018 | TC52900 | P starvation- | Similar to PSI P700 apoprotein A2 [Lotus | 0.0000 | 0.0000 | 0.0000 |
| | | upregulated | japonicus] | | | |
| AC135797 | TC44600 | N starvation- | Putative clathrin assembly protein | 0.0000 | 0.0000 | 0.0000 |
| | | upregulated | | | | |
| AC126794 | TC50291 | Drought- | Similar to GCN4-complementing protein | 0.0000 | 0.0000 | 0.0000 |
| | | specific | homolog [Arabidopsis thaliana] | | | |
| AC135605 | TC50939 | Drought- | Similar to hypothetical protein [Arabidopsis | 0.0000 | 0.0000 | 0.0000 |
| | | specific | thaliana] | | | |
| AC126787 | TC45266 | Drought- | Unknown protein (Myb domain) | 0.0000 | 0.1014 | 0.0000 |
| | | upregulated | | | | |
| AC135467 | TC45846 | Dev stem- | Similar to laccase [Liriodendron tulipifera] | 0.0000 | 0.0000 | 0.0000 |
| | | specific | | | | |
| AC121233 | TC58764 | Dev flower- | Similar to putative protein [Arabidopsis | 0.0000 | 0.0000 | 0.0000 |
| | | specific | thaliana] | | | |
| AC135317 | TC43932 | Dev seed- | Late embryogenesis abundant (LEA) protein | 0.0000 | 0.0000 | 0.0000 |
| | | specific | | | | |
| AC127429 | TC53381 | Dev seed- | Similar to dehydration-induced protein RD22- | 0.0000 | 0.0000 | 0.0000 |
| | | specific | like protein [Gossypium hirsutum] | | | |
| AC130800 | TC52422 | Dev Pod- | Putative cytochrome P450 | 0.1482 | 0.0000 | 0.0000 |
| | | upregulated | | | | |
| AC126012 | TC48419 | UV irradiation | WD40 protein | 0.2964 | 0.1014 | 0.0000 |
| AC124958 | TC56930 | Fungus- | Unknown protein | 0.0000 | 0.0000 | 0.0000 |
| | | infected leaf | | | | |
| AC135162 | TC48368 | Insect | Similar to cytochrome P450 [Pyrus communis] | 0.0000 | 0.0000 | 0.0000 |
| | | herbivory leaf | | | | |
| AC126782* | TC48366 | Insect | Similar to putative protein [Arabidopsis | 0.0000 | 0.0000 | 0.0000 |
| | | herbivory leaf | thaliana] | | | |

*No promoter sequence available.

### Example 2

In Planta Characterization of the Promoter Elements

The promoter region in SEQ ID NOs:1-26 are used for heterologous in vivo expression of a marker gene for expression to confirm the expression profile. Promoter sequences are isolated and are cloned into Hind III and Nco I digested into a binary cloning vector to insert the promoter in front of the gusA marker gene. Vectors are prepared containing each of the promoters of SEQ ID NOs:1-26 and are first transferred into Agrobacterium rhizogenes and used for hairy root transformation of M. truncatula following the procedure described by Boisson et al. (2001).

The transformed M. truncatula hairy root will show blue color after staining with GUS solution, confirming that the promoters lead to gusA expression in root. Because hairy root transformation can only be used to check gene exprsesion in root tissue, further transformation of the vectors prepared is carried out into Agrobacterium tumefaciens strain C58, and transgenic Arabidopsis plants are generated following the floral dip protocol method (Clough and Bent 1998). Staining of the transgenic Arabidopsis plants will reveal strong GUS expression in tissues corresponding to the expression profile. Tissues examined include root, leaf, cotyledon, flower organ and stem.

### REFERENCES

U.S. Pat. No. 4,237,224
U.S. Pat. No. 4,264,731
U.S. Pat. No. 4,273,875
U.S. Pat. No. 4,322,499
U.S. Pat. No. 4,336,336
U.S. Pat. No. 4,535,060
U.S. Pat. No. 5,302,523
U.S. Pat. No. 5,322,783
U.S. Pat. No. 5,384,253
U.S. Pat. No. 5,464,765
U.S. Pat. No. 5,508,184
U.S. Pat. No. 5,527,695
U.S. Pat. No. 5,538,877
U.S. Pat. No. 5,538,880
U.S. Pat. No. 5,545,818
U.S. Pat. No. 5,550,318
U.S. Pat. No. 5,563,055
U.S. Pat. No. 5,591,616
U.S. Pat. No. 5,610,042
U.S. Pat. No. 5,658,772
Abdullah et al., Biotechnology, 4:1087, 1986.
Aerts et al., Agriculture Ecosystems Environ., 75:1-12, 1999.
Ahn et al., Mol. Gen. Genet., 241:483-490, 1993.
Albrecht and Muck, Crop. Sci., 31:464-469, 1991.
Araki et al., J. Mol. Biol., 225(1):25-37, 1992.
Ausubel, Plant Physiol., 129:394-437, 2002.
Azpiroz-Leehan and Feldmann, Trends in Genetics, 13:152-156, 1997.
Bagchi et al., Toxicology, 148:187-197, 2000.
Barry and McNabb, British J. Nutrition, 81:263-272, 1999.
Bates, Mol. Biotechnol., 2(2):135-145, 1994.
Battraw and Hall, Theor. App. Genet., 82(2):161-168, 1991.
Battraw and Hall, Theor. App. Genet., 82(2):161-168, 1991.
Baulcombe, Current Opinion Plant Biol., 2:109-113, 1999.
Beardmore et al., Physiol. Plant Pathol., 22:209-220, 1983.
Bell et al., Nucleic Acids Res., 29:114-117, 2001.
Bevan et al., Bioessays, 21:110-120, 1999.
Bhattacharjee et al., J Plant Bioch. Biotech., 6(2):69-73. 1997.
Boisson et al., EMBO J., 20(5):1010-1019, 2001.
Bolivar et al., Proc. Natl. Acad. Sci. USA, 74(12):5265-5269, 1977.
Borevitz et al., Plant Cell, 12:2383-2393, 2001.
Boudet et al., New Phytologist, 129:203-236, 1995.
Bower et al., J. Plant, 2:409-416. 1992.
Broderick, J. Animal Sci., 73:2760-2773, 1995.
Buising and Benbow, Mol. Gen. Genet., 243(1):71-81, 1994.
Callis et al., Genes Dev., 1: 1183-1200, 1987.
Casa et al., Proc. Natl. Acad. Sci. USA, 90(23):11212-11216, 1993.
Cheeke, Nutrition Reports International, 13, 315-324, 1976.
Christou et al., Proc. Natl. Acad. Sci. USA, 84(12):3962-3966, 1987.
Clough and Bent, Plant J., 16(6):735-743, 1998.
Cook, Current Opinion in Plant Biology, 2, 301-304, 1999.
Coulman et al., Canadian J. Plant Sci., 80:487-491, 2000.
DE Appln. 3642 829A
De Block et al., Plant Physiol., 91:694-701, 1989.
De Block et al., The EMBO Journal, 6(9):2513-2518, 1987.
Debeaujon et al., Plant Cell, 13:853-871, 2001.
Delseny et al., Plant Physiol. Biochem., 39, 323-334, 2001.
Devic et al., Plant J, 19:387-398, 1999.
D'Halluin et al., Plant Cell, 4(12):1495-1505, 1992.
Dixon et al., Gene, 179:61-71. 1996.
Douglas et al., NZ J. Agricultural Res., 42:55-64, 1999.
Enomoto, et al., J. Bacteriol., 6(2):663-668, 1983.
European Pat. Appln. 154,204
Ewing et al., Genome Res., 9:950-959, 1999.
Fedorova et al., Plant Physiol., 130:519-537, 2002.
Fitzmaurice et al., Plant Mol. Biol., 20:177-198, 1992.
Foo et al., Phytochemistry, 54:173-181, 2000.
Fraley et al., Bio/Technology, 3:629-635, 1985.
Fromm et al., Nature, 319:791-793, 1986.
Ghosh-Biswas et al., J Biotechnol., 32(1):1-10, 1994.
Gierl and Saedler, Ann. Rev. Genetics, 23:71-85, 1989.
Goff et al., Science, 296:92-100, 2002.
Golic and Lindquist, Cell, 59:3, 499-509. 1989.
Grotewold et al., Plant Cell, 10:721-749, 1998.
Guo et al., Plant Cell, 13:73-88, 2000.
Guo et al., Transgenic Res., 10:457-464, 2001.
Hagio et al., Plant Cell Rep., 10(5):260-264, 1991.
Haseloff et al., Proc. Natl. Acad. Sci. USA, 94(6):2122-2127, 1997.
He et al., Plant Cell Reports, 14 (2-3):192-196, 1994.
Hensgens et al., Plant Mol. Biol., 22(6):1101-1127, 1993.
Hiei et al., Plant. Mol. Biol., 35(1-2):205-218, 1997.
Hou and Lin, Plant Physiology, 111:166, 1996.
Humphreys and Chapple, Current Opinion Plant Biol., 5:224-229, 2002.
Ishidia et al., Nat. Biotechnol., 14(6):745-750, 1996.
Jouanin et al., Plant Physiol., 123:1363-1373, 2000.
Journet et al., Nucleic Acids Res., 30: 5579-5592, 2002.
Kaeppler et al., Plant Cell Reports, 9:415-418, 1990.
Kaeppler et al., Plant Cell Reports, 9:415-418, 1990.
Kaeppler et al., Theor. Appl. Genet., 84(5-6):560-566, 1992.
Klee et al., Bio-Technology, 3(7):637-642, 1985.
Knittel et al., Plant Cell Reports, 14(2-3):81-86, 1994.
Koupai-Abyazani et al., J. Agric. Food Chem., 41:565-569, 1993.
Kunkel et al., Methods Enzymol., 154:367-382, 1987.
Lazzeri, Methods Mol Biol, 49:95-106, 1995.
Lazzeri, Methods Mol Biol, 49:95-106, 1995.
Lee et al., Environ. Mol. Mutagen., 13(1):54-59, 1989.
Lorz et al., Mol Gen Genet, 199:178-182, 1985.
Maeser et al, Mol. Gen. Genet., 230(1-2):170-176, 1991.
Marcotte et al., Nature, 335:454, 1988.
Marita et al., Phytochemistry, 62:53-65, 2002.
Matsumura et al., Plant J, 20:719-726, 1999.
McCabe and Martinell, Bio-Technology, 11(5):596-598, 1993.
McCormac et al., Euphytica, 99(1):17-25, 1998.
McMahon et al., Canad. J Plant Sci., 80:469-485, 2000.
Murakami et al., Mol. Gen. Genet., 205:42-50, 1986.
Nagatani et al., Biotech. Tech., 11(7):471-473, 1997.
Nesi et al., Plant Cell, 13:2099-2114, 2001.
Ogawa et al., Sci. Rep., 13:42-48, 1973.
Oldroyd and Geurts, Trends Plant Sci., 6:552-554, 2001.
Oleszek et al., J. Agric. Food Chem., 47:3685-3687. 1999.
Oleszek, Adv. Exp. Med. Biol., 405:155-170, 1996.
Omirulleh et al., Plant Mol. Biol., 21(3):415-28, 1993.
Ow et al., Science, 234:856-859, 1986.
PCT Appln. WO 92/17598
PCT Appln. WO 94/09699
PCT Appln. WO 95/06128
PCT Appln. WO 95/06128
PCT Appln. WO 97/4103
PCT Appln. WO 97/41228
Pedersen et al., Crop Sci., 7:349-352, 1967.
Piquemal et al., Plant Physiol., 130:1675-1685, 2002.
Potrykus et al., Mol. Gen. Genet., 199:183-188, 1985.
Potrykus et al., Mol. Gen. Genet., 199:183-188, 1985.
Quackenbush et al., Nucleic Acids Res., 28:141-145, 2000.
Rae et al., Australian J Plant Physiol., 28:289-297, 2001.
Ralph et al., J. Agric. Food Chem., 49:86-91, 2001.
Reichel et al., Proc. Natl. Acad. Sci. USA, 93 (12) p. 5888-5893. 1996
Rhodes et al., Methods Mol. Biol., 55:121-131, 1995.
Ritala et al., Plant Mol. Biol., 24(2):317-325, 1994.
Robbins et al., J. Exp. Bot., 54:239-248, 2003.
Rogers et al., Methods Enzymol., 153:253-277, 1987.
Ronald et al., Mol. Gen. Genet., 236:113-120, 1992.
Salamov and Solovyev, GenomeRes., 10:516-522, 2000.
Sambrook et al., In: Molecular cloning, Cold Spring Harbor Laboratory Press, Cold Spring Harbor, N.Y., 2001.
Sauer, Mol. and Cell. Biol., 7: 2087-2096. 1987.
Sheen et al., Plant Journal, 8(5):777-784, 1995.
Shirley et al., Plant J., 8:659-671, 1995.
Singsit et al., Transgenic Res., 6(2):169-176, 1997.
Small, Can. J. Bot., 74:807-822, 1996.
Smulikowska et al., J. Animal and Feed Sci., 10:511-523, 2001.
Spencer et al., Plant Molecular Biology, 18:201-210, 1992.
Stafford and Lester, Plant Physiol., 76:184-186, 1984.
Stalker et al., Science, 242:419-422, 1988.
Sutcliffe et al., Rev Drug Metab Drug Interact., 5(4):225-272, 1987.
Tanner and Kristiansen, Analytical Biochem., 209:274-277, 1993.
Thillet et al, J. Biol. Chem., 263:12500-12508, 1988.
Thompson et al., Euphytica, 85(1-3):75-80, 1995.
Thompson et al., The EMBO Journal, 6(9):2519-2523, 1987.
Tian et al., Genes Dev., 11(1):72-82, 1997.
Tingay et al., Plant J, 11(6):1369-1376, 1997.
Tomes et al., Plant. Mol. Biol., 14(2):261-268, 1990.
Tomes Tomes et al., Plant. Mol. Biol., 14(2):261-268, 1990.
Tomic et al., Nucleic Acids Res., 18(6):1656, 1990.
Torbet et al., Crop Science, 38(1):226-231, 1998.
Torbet et al., Plant Cell Reports, 14(10):635-640, 1995.
Toriyama et al., Theor Appl. Genet., 73:16, 1986.
Tsukada et al., Plant Cell Physiol., 30(4)599-604, 1989.
Uchimiya et al., Mol. Gen. Genet., 204:204, 1986.
Upender et al., Biotechniques., 18(1):29-30, 32, 1995.
Van Eck et al., Plant Cell Reports, 14(5):299-304, 1995.
Vasil et al., Plant Physiol., 91:1575-1579, 1989.
Weigel et al., Plant Physiol., 122:1003-1013, 2000.
Wu et al., Plant Physiol. Biochem., 39:917-926, 2001.
Xie et al., Science, 299:396-399, 2003.
Yamada et al., Plant Cell Rep., 4:85, 1986.
Zheng and Edwards, J. Gen. Virol., 71:1865-1868, 1990.
Zhou et al., Exp. Hematol, 21:928-933, 1993.

1. An isolated nucleic acid sequence comprising a promoter sequence operable in a plant, wherein the promoter comprises a nucleic acid sequence selected from the group consisting of SEQ ID NO:1 through SEQ ID NO:26; or a fragment thereof having promoter activity.

2. The isolated nucleic acid sequence of claim 1, further defined as operably linked to an enhancer.

3. The isolated nucleic acid sequence of claim 1, further defined as operably linked to a coding sequence.

4. A transformation construct comprising:
(a) an isolated nucleic acid sequence comprising the promoter sequence of claim 1; and
(b) a heterologous coding sequence operably linked to said promoter sequence.

5. The transformation construct of claim 4, wherein the coding sequence is operably linked to a terminator.

6. The transformation construct of claim 4, further comprising an enhancer.

7. The transformation construct of claim 4, further comprising a selectable marker.

8. The transformation construct of claim 4, further comprising at least a second promoter.

9. The transformation construct of claim 8, further comprising at least a second heterologous coding sequence operably linked to said second promoter.

10. The transformation construct of claim 4, further comprising a screenable marker.

11. A plant transformed with a selected DNA comprising the promoter sequence of claim 1.

12. The plant of claim 11, further defined as a dicotyledonous plant.

13. The plant of claim 11, further defined as a monocotyledonous plant.

14. A cell of the plant of claim 11.

15. A seed of the plant of claim 11, wherein said seed comprises said selected DNA.

16. A progeny plant of any generation of the plant of claim 11, wherein said progeny plant comprises said selected DNA.

17. A method of expressing a polypeptide in a plant cell comprising the steps of:
(a) obtaining a construct comprising the promoter of claim 1 operably linked to a heterologous coding sequence encoding a polypeptide; and
(b) transforming a recipient plant cell with the construct, wherein said recipient plant cell expresses said polypeptide.

18. The method of claim 17, wherein the plant cell is further defined as a dicotyledonous plant cell.

19. The method of claim 17, wherein the plant cell is further defined as a monocotyledonous plant cell.

20. A method of producing a plant transformed with a selected DNA comprising the promoter of claim 1 operably linked to a heterologous coding sequence, comprising:
(a) obtaining a first plant comprising said selected DNA;
(b) crossing said first plant to a second plant lacking said selected DNA; and
(c) obtaining at least a first progeny plant resulting from said crossing, wherein said progeny plant has inherited said selected DNA.

21. The method of claim 20, wherein the plant is further defined as a dicotyledonous plant.

22. The method of claim 20, wherein the progeny plant is a monocotyledonous plant.


SEQUENCE LISTING (26 sequences)

SEQ ID NO: 1 - DNA, 1500 bp, Medicago truncatula
tttcatttgtcactaacatgtttgtgttaatttatgtgataggtcctttgatgaataatcctatttgtgagatagagaaggctatcaacaagttacgaatgaagtacaagacatacatatttgagtatgtcgtcgaagatgtgggtagtccttcctcatgttgccatcgtttcatcctagggttttattgttactgttattggcaattagatatctgaaatttggtggcatgcttcagagttagaggatggagttttcaagtttttttggtttttgtttgatgatgttctaagttttttggtttagcttttgctggtctattaatggcatgatgcttcactgatgacaaataacaatgtaatgaagtgtcagttttgattttaagccatattcgtgtattgttaaagcatccaattttgtcaatcaattgatgcttcaagtatttcttttattaatgcaatgaattttcgttttaccttataatccatatgagaactttctttaattttccagttatgtgtatattgattttgtattttgttgtaaagaattaatcatagtgcactgaacctagcaaaacttgggaatttgttgatattgaagctaatttaatcaatagtcttgcttaaatggaataagaatggcgtggtactcttcaaaatatcatgattgcacatttattttccctttaccgcttcacaacatttaattcagcactactatcaattattctctatcaatgcatattcccatgcaccagcaaaagtatgtcatgtaactataaatataggtgcggtgctatcgaaaggcttgccattctcattctaaactcacttagcatcacatgtttgtatcaaaaaatggctttacgtgagtttgagagcttacttgctttgatttgcttgaaaggtcccataatctttgtttattatgtttatggtttatttttatgtgaatatgaaaaccatccagcatcaaaatctaaacgaaggaccgcatattatttaccatcatttttaccaattttcattctcaacaacaccattatttacccaagaaaataattaaacaacaacatattattttccgccaaccgttgctttttctacggacacccaaatgtaaaacacccttaattccaaagatttccaagatataaataaatacaccataaacattcttcttctaacaaagaagacctaaccattgttctaacagtaacacctaaaattcagttacctaacatgtcaaagtgaaaactgagcacaaaaactaaaatcaaaacaccagttaagaaaataattaaacaacaacacaaaattttgcattttgatgtatgaaaagctttaaaaaaagtgactatttctaaaatcacttttaaaaaaagaaacatccaaagtcatgagtgagtcaaaggggttataaacttctgcattaccctaatctttctattcttgaaattccatgagaaaataaacaacagcacataaatgggttaag

SEQ ID NO: 2 - DNA, 1500 bp, Medicago truncatula
tagtcgtcctacgtcatctgaattgactgaatgatttcacaatacttccccgattcatagaaaactgtttctttatccttgatttatctcttttgattacttagtgatagatttcttctgatttgtgttttttttttttgtgtgtgcttcaagggaacaagcagaattgaaattatatgtatggattacctgtcattggatgttggagacgtaaaagtagcatggaaaggagatgcgttcaagaaaacaaaaaacctcaaaactttgatcattaaaagtggcttctttcccaaagatcccggacatcttccaaataatttgagatttctagaatggcatagatatccttcacatgaagtttgcttaaatcatttgagtcaccttcctcattgaaggtttgagtaatagttctgtttatatcatcttgtatgtcaatgcgtcgcttttaggttattcattttttttcctttttgcagaggttcatagatatgaaagagttaaatttagactactgtggaagtctaaaacggatacttgacatatctggtctccgaaatttagaaatattttcaattaaaaaatgtataaatttaattacaattcaccattctgtttgcctaaataaacttcaagtcctagttgctgaaggttgctgcaagcttaggagtttccaaccgatgaagttaacttttctttatcaactggatctctcatgttgttctagtctcaaatattaggagagatggaaaatatcttatcacttgttttggtgggaacttcaatagaagagttgccattttcatttcgaaatctcactggtcttcatagtttacaagtagaggcacgtggaatcctttggttgtctagtagtattatcatgatgccaaaattgtcatggattactattgagggcggtctattattgccaaaccaaaatgttaagctgaattccatcctgactacaaatgtacgacaacttcggctcaaatcattgacttcctctttgcaggagaatgctactaaataagtttttattttcatgcttaaaaatttcattttacatttcatagcatattgtaatttatgttggtaatcttataaattatttatgactaacaggagctgcatatatctggaaatacgaacattcgtatggcaggatctgcaaggattctggagtggtttgagcaccaatgtatttaattctggtactccctcagtgaatttcattcattaagacccaatctgttcatcaaagactatgactgtcaaagtggtcttggacatggtagtaatatcggggtagaaccgaataatacgtatctatacgatcttcaactggaggatatgcggttcaattctatactgcacaatgcacttctgaaaaatgaatggatccatgtagagattacatgcaggtctttgatgatgacaacactcgttacagattttggaatccatgtattaaagcagaaaagtagcatggcggatattcaattc

SEQ ID NO: 3 - DNA, 1500 bp, Medicago truncatula
agaaccacttccaagctatgagttttgctttatccaccacaggttcatcgcagagtacaccatccgaaaaaataaggttgttccgagatgtccagatggtccaaacaacaccatgtcaaacaagaagcaatcctaacctaactctcttccccctcctaaccccgtgaaaacctggaactgctgggccaggccaggccaaggggaatgacaaactcccaacccaactacctagacacctgataccacaccggaaagacaggagaacaaaggagaaagaggtgcaccgaggattcagacaacgcaatacataaaacacacccccaaggccacgtcaggaagaagggcaccacgcacaagggattacgcatggtgggaatcctatcaagaataagctgccacgaaaaagctatgatcttaggcagagcccacgacttccacactctggcaacaacgtgcaaagcaggaccatctggtgcacccgaagtcggcagagcttgagccaaaaaagaataagccgacttaaccgagtagcgaccatcagagttatgagtccaagaccgcatatattccttcttcgatcgagagtgtctaggagcaaccaaaaacaactcatcgagtaggtccgactcccaaacaaacaaaggtctcctccacataaggttccaaatccggacactccccacccacatacctaactcccacacacaccctccagcctgggtaaagagttggaaaagccgactaaatctaaccctaagaatgtcattcaagcttctcttaggggtggagagtgaaatgttttctaatatgttttgaataagagagggaaatatatattttaacataagaggagagaagactgtaattcaagcatctttgaagggatgagagtgtaatttactctatttttatttccaattatctagcaaaataattataatcgaacacactttgagaaaaatagtattgcgggtctattttagtataattaaaatttgaatacatagtttttgctactttttattcacaattttattaacaaatagttttaaaccctattataaagtatgaaacaaccgaaaataagattaaaacatgtgaaaaattactaaagacaataaagtatggtttcttaaaagttaattttatactctaaattatacaattaacaacataatatttaaatttaattattattgtcatttaaattataaataaattaatttattttacttaaatgatggttcaaagctagtatatattataaaaatatttatttatcttaaaataaattaaagtgtagtatatatttaattattaagggtgggagttgtaattcaagcattttatatgagaggggagtataaattttatttttcaagggaggagaatgtacattaaataaagagtgtaattcaagcttctcttaaaggaggggagtgtaatttactcaaaagttaaattatacaacaaaaaaaaaaaacttaccttaaaacaaca

SEQ ID NO: 4 - DNA, 1500 bp, Medicago truncatula
attctaaagaaaagaccatgggtccgaacaaatctaacatagcaagtttagtgtcttcacctaactaaaccacactcatacaataaatgaacatgatactttttctcattttattttcatgttttctacttgtctaccaataatcaatgaaaattcaataaacaaaagccgtacaaaatgttgtggccacaaatttttgagatgatgaaaattgaaagccaaacaaaagaaaattatgtggtgtagaatgatgtttatccctttttgttaatgtaacgtgtcatgattcatatagcttggcggcaagtggcattgacgtggctaaaagcgcaatggaagaaagtgatgctctctatttctcgttctttatcatccacaagctttggcctacccttcactttacactttttctttgtcatcgttgaatttttttaaaaagcactagatatttaaacactttttctagtacacataaatatcctctattttaattttgtaagaataataagaatgatgataataacagctcataataattgattttttgaattaatttgattggtacactgacccacttgtttagcagcattgtcacgctattttatagcttcatttcccactcgctcacagttttttttttgttacaatgctaaacaaaaaaaaataacaatttctcatattctctccgtcctaaattgtatgatattttggccatttcacacatattaaaaaatgtaattaatattgtgtggggaagagaaattatgagttgttttacaaaattatccctaataaatggtatggaaaagataaatgaaataattgaaagaagagagagtaattaatagttaaggatataataggaaaagtaacattaatatttcattgggattgtaaagcgacatataatttaggataatttttttctccaaaacgacatacaatttgggacggagggagtaataaataatgatccattaattaatatgataacaaatcgtttgatgtatgcataaaataattgtaacatgatatactaattaaatcttatcaatataagctaggggtttaatattgtcaaatacgtgtctaagatttaatattgttttagtaaaaaaagatttattataatattatgtttgattttttttatttttttaagcaaatgtttgatattttaaatgcgagtaaaagtgaatgttaaaagatatttatggttaatgttttgagtcatttaaattattcaaagtctaatatagtaatccacataacttctcatatccaacatattattgttaggtgaggtccatgttataaattttcaaacaaaacatgagtatattgcagaatccaaaatagaaaaggtactttgtatctagtcaaattataatgatatttagaaagtcacagtcattaccatataaggtggattttatctcaagatttgctgcaaaagtatgtataataaagaacatcaacatagcatatattttgcattgt

SEQ ID NO: 5 - DNA, 1500 bp, Medicago truncatula
tggtgatgcatctcgtttctcctcttctttgccatattgtggtccaccatgcttggtggttaattataaaatattaatgtgagataaagttgtgggacctaacatgagttttttaaagttcaccattgaagaaaaatacgagtgagttgttccacatgtgacataatggaacccacaacttaataattatttgagttcatcattagaggtgctcttagtagatgagtttcacaaaaaatcctattaacactaatggagcctaaaatatcaaaaccaggaagcaaatatggcatgttaatgttaccttttaccaatctaatttggctatattctcaactctcaccagaaagagaatacaactaatgtccaaatatctagttagaataataaccgtcaagcaattttttaatgtaggagagatgtatcaccataaatagaatgatcatttgttaagaaactcatatggtagacgttcatgaaatttgggttatcaaacaagagtgaaccatggtttccatacacatcctaatcacttcaaagatttaatagataatttcgagagaatagagaatgcaatttcatcgtgtatatgttggctatttattaccctttccttttgatcatttgccggaaccacaacagtgcatctctccatcttgattccacgaaggagccttaatttttaaaaactaaatcaaacaggcccttaattttcttgttagaaaaacacacttaagtgattttcaagcatcatctcgacctcagagtcttgagtcgaaagatattaaagctttaagaggatgttttgttgcctgagtgacacattgataataatttatctgaatattctaattagaagccatgacggaagcttggactcaatttcaagccttcaattgaggagtaatttttgtttgacataaagatttggacactttatttatgaaaaacacacaaaaaaagcacactttatttctaaaaaataaatatcatttaattattattattatttttgtatgtgtccgaaaaatgtatccaaaccatagataaatttttcgcggatcttcaagacaaaatattgtcataatatcattctgaagtaaggaaccatgtccacataaaagtatacaagttccattagactacttaacaaaaaaaaagtatttacgctataaagtttgagaagctctttgatgtgttgtcaaatgtatctaaagttttaatagaatgtacataagttttatgcaacaaaaagtgtaagtaccaaaggatcaaattatcctagtttggatacatcgccaataaatgaaacaaaaaacatagtagcttaaggccggccatttgataccatcacatattctcctctcatcccacgtccaaaccaaaacatagatatggacacttacaacattgtactaacatcaatttttgaaggaacatacaaagaaaacctagtaaacatcgtagtctaatttttttatttatctaaactaaactttc

SEQ ID NO: 6 - DNA, 1186 bp, Medicago truncatula
  modified_base (55)..(58): n = a, c, g and/or t/u
cccccgccccccccccccccccccccccgcctccccggcaagggtggaggcgggnanncgattagattcgagctcggtaccctaaatttgagaggaatttctcccacccaaatgtcatgccagaataaagcatctctaccatcacccacaatccgacgagagttatcttcaaaccatcttccaacaccctccccaaggccctcacgcacaccacacatctcccgccaccaaaccgaacaacggctaccctcatccttcaacctcccccctcctcattgtaacgagcttttaaaactcgatatcacaacccctccttctccaccaacatcctccaacaccacttttccatcaaagataaattaaaagctcccaccctcctcacccccaacccttcatcctcctttagagtacaaatagacttccaatttatctatgctattttcccaattgtcaatttgttacaagggaggattattggcacaatatgttttagcatatgggtgcctcaacatagctatgaaaatacatacgtttctcttgcattggattaaaattagacaaactttatgtaagactaaattttatttttttttacggatgtaagactaaaattaaaaagtcgtatttttttttacaagaaaaagtcttaattttatagagactataaacatatttgaacatttatcttatatattgttttactgcaatatatttttgttgagtttgaaatttttatgtatttcttatggtgtagaattctttacattttatacaagtaggaaattttaaaaaacctacacagtctttggttatattttttcaactttacttttttttttcacctcgtttttcaacattattgtgattagcattttttttattagattaaacaatgattacgttgtaattaaaaaaaactaaggattacattgattgcatggaataatatttatattaatgttcagtttattttattttaatttaatttttaaaaatttattacgtctatttaattaaagtttgtttgaataatttttttaaaagattcttgtctcttaataatttcaatggtcacccccagaagccaatagattctttgaaaaaacaagcacatgaatttacttcatttctttccacctcaaagttgtctatataaacaagtatttaatgtaaaattataatatcg

SEQ ID NO: 7 - DNA, 1500 bp, Medicago truncatula
ttgcaccatcaaacatatagatattatttaagtggtttagctttaagtggagtaaactttttgcgatgaagtatgatagttcttttgaactattaatattgttggtgtatacaattagtataatatgtatcattttcaattaaggtggatatttttggagttggttagaatgaaagaacacccacactggaatggatccagaaaatgtatcgaacggagactgattttttctaataataaatataatataaatttaaaaaatggaaaagggaaatttttaattaattaaagtttcactagctaatggtcattttaatagttattctattctattattttaaagttttccaacttaaattctagtgttaaatttgggctccttaatttacctatgttgttttcaaacaaccacactttgttacaagggagaaatataggaaggattgtaccaaaaaaaaaaattataggaagaaaatggtgagagtatggtggagtttttacttgtaaaattactattaaaattttataaacttgaaaattcgtgattttgtagggacaaaaaaatataattagcccttttttatatttcaaagaaattttaaaaattacgcaatattgttttaacgttacttttttttttttacaagttgttttaacgttactattgttaatgttttgaattcgattaactaataataacgtttgtttaaatggcttgtaaaaaaaaaacgtttgtttaaatggaataatatatatatatattaatgtttagtttattttaaaaaatttaaaagttgttactttatttaataatttttttttacaaaaataaacgatattcattcattcaaattgatagagtacatcgatacaatacaaatccaaattcgctaaaaacaaaaaggatgaatttgtgaacaaactcacaagtcacaatatccaagttaatagcataaaacgacaaagtacttatgcctacaaatatgactaatatgactatattgatgtctctggaatactcatgtctccagatctgtaacgttgacgacgacaagtcattgtcattgatcggatctgcacttgctcaaagttaatctcaacctgaatcaaataaacaccgcactaagacaggaaataaaaaacacaccgcacaaagacgggaaatcaaaacaaacagagtcgttcaaagacgacgaaatcacaaaatcaaacgaaagaaaacagaaaatatgtgaaatcacttattcaattaaaatagaagaaaaaacaattcgaatgggtgatttttgggtcaaaattgaccctaaatcatgaagaagaaaagtttatttaataaaaaatattattaagtttttggaaagattcttgtctcttaataattttaatggtcaccgccacaagccaatagattctttaaaaaaaaaacaagcacattaatatactacatgtgtttcacccttcaagttctctatataaacaagtgtcatatgtaaaattatc

SEQ ID NO: 8 - DNA, 1500 bp, Medicago truncatula
ttgttgtattagttttttttgccgtcagaagaaatctataaaattcctgctttcttactaaatgagcttccaatgacgattcaaccggaaacgaacttcaaatttaaaagagatgaaaaaaaaattacagacaattttttatgaagaagaagtgcttccacttcaaacaataataggttatcttggtaaaaaatagaggcaaatgtcagccagcaaccatacttctctaatctattccattttcatcttcaatctcacgaataagtaattattttccattttcctcttcattttgagtttaactattttgatactgcttggatcttagttgaagaaatgagaagaggaaaaatgggtgaatctgttgttaagggcagaaaaaaaattcgattgaacaaaaaagagtgtaaaagataaaggaaaaaatatagtgtagacagtccatgataaaattaatactcctctttaatctaatggacaaaaaaaactttcccatagttgaaaacaattcccctttcccatgttaaactcaacccctctctatttatatttgtgttgttggtggggtttagcatgggaatgagaagttcttttccatgatggaaaagtttaacaacaaccatagatttaataattttttatgtgaatgacctagatcctgataaaggctgaaactttttggtggttattgtgtaataaaaaaagctgatcccaccaaatgattgaataacaattttccatttatttgattaataaaaaataataaaaactaacaacttcatcatcatccctcgttatcttaaactaagagaactcatacacatgacccttggagaactccatccattcatcttaatctcttattaaggttatttaaaaataattgcaaaagcaattgaactaggttgaaatacgattatttcgatatcatttgaaaaaaaaaattaaagtgtttttgttcttgaacgattaatcactgatttatgcttcaaatataaattatctcatgtataatattaatataacaatattattctctatgcataatttatttcattattatagtaaatataatctatgtacatataatatcatataaattattgttaatagagaaaaaatcattgtgtgtaccctacatttaaggatgaaaataggctaagttgaactagattttgtacggtctaagtctggtatgtcaaaaaaattaaggtctaaatctggtttgcgggctttcataagcttgttttctatgcttgatatggtatgttagtctacttaaaaacatgtttgacattaacatttttaaataaataacatggttaaatatattttggatccctttacatatatcaactttttatttcagtctctctaaaatttcccttcgacttttagtccctataaaaatttcaatcactacttttggtccctagttttaattcaactcatatgtagcctttgtatttttaatgaaattgtgcatatatgtgtagaata

SEQ ID NO: 9 - DNA, 1500 bp, Medicago truncatula
gagtattgaaactattattaccattcgtggtttcagttacaccatttatgctcctaaatctttgcgtgattgaagtattctttttcccttagattagaactagtggctctagatgccaatttgataatgtaacttaagtttcttgcaatgcaagcaagagtttctatgaaaattagggttgagaaattgagagaactgagagaaagaagaagagtaattgattgttgtaaaattgtattctcattaagttgtgaaaaaatatttacaacatgcttataaagagcttatttacatagcttgcttagtaagcaatcaaaacaaactgaactgattttgctaactaatagccaagtcttccaacaaacattctaaccatgatttaatctcaactaacttgagttttttttttttctttctaaagatcaactaacttgagttaaaaccatctaacttgaattaaaactaacatgatctgtcctagtgtttttcctgtcatttgggcgagttttagttattcctcgaaaaaaagtttgagattcttcgttgccatttgaatattatttgattttagagcttagtatacaaattagcttatgtggggcaccgagtcaggattttttttattataaaaagtgcagtctggtgcactaaagcttccgcatacgcaaagtcaagaagaggtctcgacatttggcgtatcgtacgcaattctatcctattttttacataaaacattgtttctagaacttgaacaaatggcctttcaattatgttatgtggcatttgaaaattatcttagtcaatttgtttcataaataattcaaatgtatatatatatatatattataattctctctcttaccctttatcacatccatttccaaagtttgaattaaaaaaatcatcactgaactcatgaaaaaaatgcataaaaaaatcccatcaaacaaattcaacgaaaatctacattaaacaaacaaaagacttgaaatctacattaagaaattaaacggacgttcctaggtatgcaagctttgaaaatatcatcaaacaaaagactctgaaactcactagggggattctccataatatgaaaatctagaaaatccatcaaaatgttgaaattagcgagccaatctacacttcttttatcttcacgtcaagaataggtaattttgacagtccaactcaaactcaaatttgactaatgctactactatattactcccttggtctactatgtatactattcatacaagatatttgattatatttttctattaatatataagataaagatgttattagatttttcttgacaaaatacttacaaaatatttactttaattattttttgttaatctaaaaaattactcattccggttacatttataagcaaaaaatcacattttagatacattgaataattaatgtatccggccacatttataggttagatacattaactattcaatatatctaaagagtagttttttttac

SEQ ID NO: 10 - DNA, 1500 bp, Medicago truncatula
attataagacctttttgagataaaaaattgtccctttttataagatccctttggtatttccaattatattaattatttctttaccaacatacccttatttattttgcatttttttcttcaatcaataataaatattccctccgtctctatatctaagcacccatttgattttatttttgtccataaatataaaccccttttcaaattactagatgcatttattatttttttcctaaacatacccttaattaatactaccatcttgtcttgaaatatgaaaagtcaaatttaatacacattcaagcaaatgacaatttcataatattaacacaaaaaacagacacattaattacattgacaacttttcttaagaaatgtaaaaaagacaatgggtgcctacatggtgtttgtttcaaggttaagaatgatattcccgggaatacatggctagggaatgattatacccatgtttgttatgaggttgagaaaaaaatattcctgggtataaaggttcctgggaatggagtaacttcccataacttccattattcccatgtaaagagatgggaatgtgcattcccatgatattatgcatagatttaaaaaaaaaaaattgtaacttccaatttttccaggaatgtcaagttatttgccaaacactttttttaacaaatacccaagaatcaaattttcatattttcattcccgggtatgttattcctggaaatagtttgtgtttcccaaaaacaaacgccccctagggatggagggagtattaggctgaaaacactaattaactactctctctcttaaggataaaattgtaaaaaccatagtaattatatacaaatttaataccacaaccaactttcttaatctccgtaattttttcaaaaggctcatatatttagggacggaggtagtatatatgtaaaacaaagatatttataaataatgtcattatcattaaaggtatttataaattctagtttcctacaattcaataaagataattttttttttataaacaccaagttatacgaacaagtcatccggtttaatccaattgagtcatgcggttcaacctgtgacccaatggtttcgaccaatgactcagtgatccactagccttactgagtcgatcagtgagccagtttttaaaactatgcttacaagaaagattatttattgtatcaacgcataggcttgtctttactttttaaagtaaaatgtatttctttaaattttctagcaggtcatttttatagatgaaacaaaatatgtgacgagatatctgatgttagatcgatgcacataattggacaagaaagaaacttgaatttagggagtaaagattagggcacgtaattgagacactaccagtaaaattaacgttagtggcactcaaaatttagaagaccatgggaccttacatagttggtaaaaatcaaaactcttgtatgacatacgaccaaacattaattcgtactcagttcttgt

SEQ ID NO: 11 - DNA, 1500 bp, Medicago truncatula
atttgtgcagattatattgctaagcttggagctggcagtcaagacgcgttttggtcatcgcggagcatcaagatgcaattctcctccacctgctaggcgatgctagtgggaaattctttactagatagttgttgattttctttttgtttctttgtttctcttccatagttgtttattttctttttgtttgtttgtccattttcccatttgtagaaaaaaaattgtttttaatgtttaaattatcataatgggttttaacatgtgttttttttttttctccgaaaggatatctagatttgtttatcttctcctttatttgtttatattatccttcatttattttagattaaattgatgataaaaatattaaattagcaaactagcatagacaaataaatattaaattgataatatatacaaatattaaaaggaaatgcttaaaaagatatttgtgagtcccttaaaaaaagaagatatttgtgaagactatcaacgaattcataattctctcgacgtaaccaaagacttatcatcatgcagtttgattagtcatctcaaagaccacataaagttgaagtttatttgagcgaacacatgcacaataatcttttttcctagagttggatggattaacaattgtggatctcgtaataactttggttatatgtggatgaaattagtttagttgttagaatttgaatttcatacatattgataattcatcttgaatataccgttaacgctaatactaatctcaaaataaaaatgaggcatgtcccaacttatatctttgttattgaatcattttagtattatttttttgttacatcttgattttagtatttgaatgtataaaacttcttttacttaaaaaattagaatttcaattatgaactttcttttatcaagtagttgaatggttaaaattcactttcaaaacaaataagtaaaatgtttgaagttcgaactctaattccacatatatcatgcaatgtatttatcaattccgaactttctaaacactatcaaatccaaccgtttaaatcacacttgcctacagtgaaaaatactctattaccttctgacccatttagtggatcacgtattacgtacttatctcattctttctagtataattccgttataaaaaataaaaataaaaaaaagtaaaactttactttctgaggtgatcccctaagcttgttgctcccggcaccagtcggtggaggtgtagaaaagaaagagaaggataacgtgtgtttagttctagagatcaccagcttaatctgtctttagaattcgtgttctgcatgcatacaaaattggtcttctatgtgtcatgctgcatgcactttgctgcttcaaatgccacctgtacctccaaacatggaacaatcatctttcccttgcatgacaagtgtaaattctccattcctctttttgtatccattaatttcttctacatgccctgtatatatacacactgcaataaattatcac

SEQ ID NO: 12 - DNA, 1500 bp, Medicago truncatula
caagcttttaacaatatggccgtgatctgtcattacgtgcgactatctccactatagaaaaaaagaaaagaacgaacaactccactaatactaataaaaaaataaatactagacaaaaaaaatgggctttctacatatatatatcgttcgaaacaacgatttttatgagctgtagcaaagaaagaaacttcatagaagtcaaaatatgaagaaatagaatatatacgcctagatactttttttctatggataaaagatctaattgatagaggaagcaccgtaaagatcaattaacaaggtttttggccgatacaacaagaactgcttacttatatgatgatagattaggaatctacttatgtaataaagtggatcccctaaggttgtgagcagcggtgtagtatcagatcccaaagatagtaagtcttttcttatgaagaaaagtctttctcaaagattctatagaaattgatatatcatatatgaaagtatatgatatatgaaatcgagatagttacctttcagaaaatttcaataagaacgagcattagtgttaatgtcgatactttattcttcggaaggtaggaaaaaagataaaactaaaaacaaaggattaaattcgtaatccaaattcaagtttgaaactcatgtaattaacctcttttcttgttcttaactccagaaaaaatggaagataaaaagaattgactgttgagccgtatgagtcaggaaactctcaagtacggttctaagggaaggaatttactcacctattccgaccgcggagaacaggccattcgacagggagattctgaaattgcggaatcttggtttgatcaagccgctgaatattggaaacaagctatagcccttacccctgggaattatattgaagcacagaattggttgaagatcacagggcgttttgaataagaacactctgtttattattttcttttttttattctaatattctatattttctataatttttctataattctctttatattctattctctattctctatagagaatagaatataaagagaatacggatcattcattttctaaatataaagcaacaaacaatcaaaagattttcttatatcccttctaaaatagttctatttcatctgatatttcgttatgttccttagtaatgctaatataactgctcacgtgatttgaaataaagtacctaagaatatcttctctgtaagatatgcaagaaagacaaaaaatttcatttgttccatctaggaatctctaagtgaaatatgaatagactaggttgcacaaaaaattctttaacttcattaaattcaaagttcagaaaaggttttagaattaaaaaaaagggtccgttgagcgccttactactatgtcataatagatccgaacacttgccccggattaacttccggatcataattgttctagtgaataactaaagaaaatatagaataaatagatgggagatagaagagaaagttattc

SEQ ID NO: 13 - DNA, 1500 bp, Medicago truncatula
tatacttcttttcgtacatattgcattgttagaaccgcaactcaatacaaaagattaacaaaataaaaaatgcaaacaatgaacattagatacatacttgttaacaatcttcggtcaattgtgtctgtctataaaaccctagctacatagcaatcacaatttcgttttattgttagcgaaatattggagtttagtgcacaacttatatttaagtactacagtctataaccacatgggttgatatggtgatattgacttgggaccggaatggattgtttcgattttggaaggaaaaatcatttgatccaatcttttcgggtttgttatttgttcatccaattagtttggttttaaaatcaaatccaatccaaattttttcggtttgtatttgattggttttcggttttcattgttgtatattttttttattttcttttaagaaaatatattattttatgacatttcataataaggtaaggttacgaaaaagatgaataagtagaaaataaaagtaacataaagcagataaaatagtaggttaaaaaaaatagttacataatcgtaaattaatactctttccgttacaaaatataagcaaaattcactttttaggttcattcatttaatgatgtatgtggtcctaaaaagtgaattttgcttatattttgagacggagggagtaatatttcttaaaagaacgaaaaaaaagtcagcattattatagaacaaatctaaaaattagttacaaaattgtaaattaaagaaacatttctaaaaaattcatataataatatatatacaagtataaatatttcggtttggattggatttgatcggtttatactaaggaaaccagaaaccaattcatttaaaatcgcggtatttttttatttcggtttttggtttgttttggtttttagttttttggttcggttattgcggtttgcattggattggattgaacatgaacacccctacctgaaaatgtgctcctcttcgaggtctcaactttgattctctctggtttcaatttaggtgagctaatttaacttcttcaaaaaaaagtcgagactctggaatgtgctcatttatgttaacaacttcacttttatgcttttcaaattatttgacaagttgccaacgcatgaacaatttatatttgtaatgacgtccttatggagtttgtggaatagtagaaacacaaagcaacatgatgtcattgacacctctctcgcttccattgttacctgagcaaaggacatacaacaatgagagttgcatgcaaagaacaaaactatcggtacacaactgaaatcatcttcatacttggatcaaaccctgcgtgatcataactctttcaacaacaactatgtctcttagtatattatgtgttttagagactacgaggcttcttttacttgaaaaatcatattatttttactttttgacaccatcctaaaagcctaaaccattggcttgcgagaagccatcaaaggggcc

SEQ ID NO: 14 - DNA, 1500 bp, Medicago truncatula
aggattaaatcagaaaaagaaataagataaataacaaaattgttacataaaattaagttaatgatttttttttttgaaggaaaattaagttaatgattgaattgtgtatttttgcctaaaattaatactccttcaaatttctttaattaaactcaagtaagactgatctaataatagatgataatctaaaagttttcagaatatatgaaaaggaatatataacaaggattgacaaaataaacacatgaaagttaaacaaatcaataacaaaaatatattaaaaaattacaataatttaaaattaagattgaacttaaacctttatgacaaacattgtctccccatcctcgtgagcttaactcagctggttggacaatgcataatatatgtaaggtttgaggttcgaatttcggatacaacaaaaaaataaaattgtcccccaattttttttccatttttttaataatatatatgagcatagatgatttgagtctggttgagataccttctattctttttaataacatcatgctcttaatttagaaagaaaaaacaaacaaacattgtcatatgcatctgttcgacgacatatgttgaatattaaatgaaatacattttgaatgttgaataattacacataaattttatgtcatttgaatgttgaatataattcagagtgttgaatttgttttgttcgaatgtttttaagtacgtttgagtgtgttatgacaatgcattgtgtcagatgcctaagaccgataataaattgaaccaacttgagaataggtcgatactcgattcaattggttcatgagttagctagatacaataaaattatatcaaatcaccgtaataaaaatttcaaacttaaacaaaaagagtaaatcaaaacaaaaatattttacaaggtgtaaaaccgaaaatgacctatatcacagggggtaaaacactattaatcctgtaaattattgatttaaaccacttgcttttcaattttggggataagagacccgaaacaaagatcaattttttttctttctttcactaaattagaacttaacttttggtttaaagtataagttgtttaaaatatcaaaataactttgtataaattaaatgccgtatacgacaaaaagttgtttaataccttaaaatttaacatgtaatgttcaatccaactttttttacttttaagcacttttaaaactcttatcttcaacattttatttagttgagaaatgcctaagaatgcctcttggtcatttgttaaataaactagcatagaagtgttacgttagaaagtggtggaaaatgatatattaaactttttgaaaaaaaaaaaaaaattatgattttcaactatttttccatgtggaaaatgacgtcgttatgattgagattgaactttattgacatgtaagaccatctacaatggatggttcttctcctgtttagcacgaagctaaataaacatcccattgtaacatcaactaattgaat

SEQ ID NO: 15 - DNA, 1500 bp, Medicago truncatula
aagttacaacccccaaaaattgagaggttagacatccctggtttcagtcaagggttaaagagttggagttcctgaattcaaattcgaacaaagattaaacattaatataacaattaactactaatatctgataggtagtgtaggttttagacattgaagtgtgttcctctccgtgttgggttgccctatgtgccaacaatttatatgtttgatcggtccataattttgtttggtctaacttcaactggaccatgactagtagaatatgtatttgatccccctgattaacatgtctttgagttgaataccaagttttgtaaaaaaaaaaagaagcaattacttgttctttttattctcgacaacaaatctataatctacaactctgattaaagcagtacagatctcgatagtttcacacaatttgagatgcttgaacaatccatatacaatgacatcagagtttctaatcatttgttaagagtgcttgaattttttactaattaaaataatgattataaaaaatctattttttaagcaaataggatgcagatggatctgccacacctcaagtagattaaaaaaggaagaaaaaatatagaaaattaaagcgacataaaacctgacccaatgcaaacagaaactaacaatgagccactaatatcaaacggaacagtcaaccacacaccaattgcacaatctagcgaacccacccaaattgcaaaccaacagaggtgtgtgagttgacaagcatgtatataagttattgatataggcgaatgagtctctctaccaacacggtaacaaatccacgcaccaatagtttacatagacaaacaacaactaatccacatagaaacacatgaagttcatatcgaatccaaccatctacgaaaagacaaaaacttaacaaatgttcgccatggtggcgccataagtttttgactgtaaatcgtaactagactagtctagaaccacccaaaatcgcgtcaacatatcgtatttagttatcagcaaaaacaaagggtctacgatgacagaccaacctacacatgtgaccgcttgaaataggccaccggaaacactaattaatttgcgcgagtttataatttctctgtcgtttcatatataaaaaaaacctaactaattccaacattatttttgaatgagctaattaattccaatactattctactctctccgtttcaaaatataagtaaattttattttttaggttcattcaattaatgatgtatgtggtctataatatgaaccacatacatcattaattgaatgaatctaaaaaataaaatttgcttatattttgagacggagggagtatttgtattatttttccacattaataattaatttaattaaaagaaaataataacgtgactagaaaatgaggagaaggtgggatttgtgattgtgtgagagtcagctcagatctgagtctgaaaacggctataaataacaggaaagacacacttaacacctcgc

SEQ ID NO: 16 - DNA, 1500 bp, Medicago truncatula
tttagctgatccacgttagggctcgggtgggccacaacccactcgaacaaaaaaaaaaaaaagaaaaattattaaaatattcttgacttctaaataaaatcactttgtggcccaccctcttctccatcctggtatatgttactatcatacttttaagttacttaaaatgtttagataattattttgaattttaaggtatgcaatttttttttattgttaactttgtgtttaatgcttattaaaacataaattttgtgtgtggtattagtttatgttagtggccaatttttttttgagcaaggagagatgacagacatcaagagacatcatcgacatcccaactagattggcaccctgagagacctccatcctatgccgccaaactctaaaaatttattaaaaaaaggggaaaaataaatacaaaggcggggggactaggccaaaacccaccaaagaaaaaaaaaacaagagaaaaataaaacaacaaaaaacgcccagcaaaacagactatggaaatctgtagttgggaacaccacatccatccctaaagaaatcaactttcaactcattagggagagcagacagccaaaccgaaccctgcacttaatgacccatgctagcaagcttgtccgtaaaaggccacgacccatggaaacaacctctaacattccaatagaggaaattcattaagcaggagtgtgcttgtcaccccgagcaggggttttagaaggctgttttggcaatacttgttgcaatttaagtttttgcttctgcttccttgtaagtaccggcatgaagtcttcattagcacactgtgcatcatactcacgaactctgtcccataaatcaaggccgtgttgaatgttcttactaggatggacatgttgttgttgcaaaacgacaacctcttgctcctctgaatttgacgtgttctcaatagctgcaagagtcttcgcaacaacaacaaccggtgacactgggcgtgttgttgccaatttgattttttttttctctagctctaccaccgttcaaactctatttgaaataaagtctaaaaaataagttatgagctagtaaaatacgtaataagtgcatggtaaaaagattatgaccaaacatgtctttctttgtcatacaagcttataagctataaactataagctcgtttcatatctttgccaaacagacctttaaagtctttgtcaagtaaataattcctcattggagaaactcttaccccttcatgattttgtatataaatggtgtgtgagtccagttacgggtcttcattctatcttgcttacagttcctcaacttcacaacctttattttcacttaccatttatttagttggagctcaagttaattttaactatgaagtttgcttacatatccatctttatatctctcagtgtaagcacccgcttccaaatttctaatttatcaagtgtcaacatatacttttccccatatattacctttttagcattatttcatttc

SEQ ID NO: 17 - DNA, 1500 bp, Medicago truncatula
acaatagtagcaaatcaccatctggttcatggagtatgccatcttcctcaagaatagatacaaatcgagccacaattccagtacctgattgtaactgtggcgcttgaggacaaccatgtttaaggcaaacacgacagagaaccctgggagaccattctacacatgtccatttcacaaggtatatttgaaagaaaaattgaccactaaaattgtatctacacattatacaccttatattccattgttgtttatgatttctccatttatgcaattgtagaatgatccccgaaattgtgggtattttatttggcttgatgaatgggaagaaatctttggggcagaagaagttgtaaatgcagaggagaaacatttaagtagaaatgatgaaagagagcctctaaatgaaagacttcttgatgggagagaggttggcaagggaaatggagatgagggaaagatgaagatgaaatgggtgatgataaaggagggacgaaaaattactgttgcaaactatctactaacccttcatattcatccctgccagcgtggaaagcctcctttgcaggtgatatggcaagaccagcgtccagatcagcagagttaacgctcagttaacgttttggatgggaatgactaacggaagagactttcgaggagttttttttttgcgtttccttgttctagggatgtgcctgatagcgaggcacactttcagggacccaaatgagggtttactttttaaaaatgtctctaaacccactaaagcgttgaagtggcatgcttttatccacgtgtcagttgttgagaatgcaactttgccatgtggcaggccgcataatattgaaaaaaagaatttttaaaattctagaaattgaccgaaaataaattttcagattatttcattttcaaaaattaaaataaaatttctgaaaattagataaatcgtttttgaaaatgatataatctaaaaatatttgaaaaacaaatctgattatttttgtttagaaacttaaatttaattttttgaaaataatatttgaaaaacttttttttgaaaacgaatctgaaaataaataaaaaacatttgaattttttttaaaattagatttttagaaaaaattctgaaattaagatttcaaaagttaaaatgaaatcatttttttcttcaagattatataattttcaaaaatttattttcatattttaaatttaatcgaaaaaaattaatatttagatctaaaaatttatataatcatcgaaaattttattttcatatttaaaaatttgaattaaataaaattatataatctatttattttttagtttattaagttattttaaaatatatatatatatatatattttatcatttacaattataataactatattttatttctcgaaagtgttggctaagaaatgaaatgatatatctttttcgaaagaatgaaataatatttttttaaatataaaagtcgacaaatatataatatataaaac

SEQ ID NO: 18 - DNA, 1500 bp, Medicago truncatula
aatgagatgaggatggacatgtttgttttatacgagctatgtcttcgagtacaaaaaggaaactggaacaagaatgactatttgaatttaaaccaaatattggaaacgcctaaagatgcgccaagattgtctaagattctagttataggtgtatgctcagcctctctcatgatggagagctgatgatgacctaacatagtaatattcttgcatgttaagtactatgatagtgctccgaaaggtgtgtcctctcatgcttaacatcatgattgggttgaaagatcccaaatgccaccatgttgccacagagccatagaaagttatatctctgtatatagaagcaaatctctgtcgtaaccgattcattatttaaatcacatttttacctttaagaacttataaacctttttaactgctttcctaaaccgttattgcttttatctttattctattgttgtgacattaaatgatttattttattttcttacaaacaagattattgttaaaagtactacatgaagctataacgccccaatctttgtgggttcaatcttttataatattacttagacaatgtatacctgtatttagttaaaccacttcaaaatacacagtgtcttcaggcgtaacaaaaggtatctcagtaaatgatgataattttaggaatgaaatcaaccaaaataatgatttttttaagaaccgcttacaattcaaatgtgacaatcaaattgaaatggataaagtattcactccgtctcaaattacaagtcattttggtcattttacgcgtattaagaaatgtaattaattttgtgtgtaaaagatatattatgagttgttttacaaaaatgcccttaaaaaacgggtaagagaaatataaattaaataatgaaaagtataatagaaaaaataacattaatgtttttataagtattctaagcgacttataatttaagatttttttttttaaagcgacttataatttcagatagacatagttatatttttgctaaagtacttttaatatgtaatgcatttatcagtatcataagtgtcaagtgtatttatcagtatcaaattgtaaagtagaagatattgaattaaaagttatacaaataatattaattaaagatatgattgaaaagagtaattaaagttgcattgaaaattgaaatagttaattattttgtgacatatatttgttgcaaaaatatcaattactttcagatggaggaagcataaataagacaaagttacacctgtggtccattaattttgaaacacccttactagaaatcacctaggatatctagcatgtgtgttaaacttggcgtcggatacattaaaattataataaatagatattctctgaacaaatcatagaatttcttgcatgttacctccgaaatagttccactgacgtgtactctatatggtcaagctacttcttcttcttcatcacgaaccatcttgtcgtataataacttgtatatctgaaat

SEQ ID NO: 19 - DNA, 1500 bp, Medicago truncatula
gcacaagtcactcaagtggtgcatggtgcaagataaatttcttaatgattatcgatacacttggaggttatagattttcaccataggaatcaagaccatgttgtttcgatgttccttatacagcagcagctatttgaagtcggatcttaaactgataaaaatcgtctaacatcatcaaattaaaacaaaaaatacaatagcatactgatggataaaagatttggggtttaaagaaagttttgaaacccaagttgaatcaattgaagattgcaacagtaacaagtgctgctgggtatgttaacatgatgaaaattgctgtatacacattttataccgaaattaagaaccggctgtagttaactacagaagttccttcggcagttaactgctgaggagctcaaaaatgccaaattatttccccctacttgttgagctagcgtatatatgtgtggtttgtttgtcgtggaatcgaaacttaagtgcctaaaggtcacaacactattataacttaactaccaaattccatttgtttccgtttgtttttttttccctcttttgcctctcaaggtcacaaaaaacggctaaaagtaagaaaaattatctttctttggtgccactcttttgaggcatggttcacaatccttacctacccttgtaaacaaatatttcaaattcaaaccctttgaatttaaagctagcttacctataaattgaggataatcaattattatatcctacatcctcaactaaaaaaacaatatctagaggacaggtttaagtttctaaagctagacacacattttctatatatcttctactattttaatttctaaatgaggcacttaagtttcagttaaaacgcatacgctaactcaacaagtagggaaaaaaattggcagttttgagctcctcggcacttaagtgtcaatggtttttttttcttcatcggcagttaactcccgaagtttccttcggcagttaactgcagccggttcttaatttcggtagttaactgtcacggtggcattttcgtcttttacttgtgtttttcagccttataacatgtgtatacagcaattttctaacatgattgggtttgaatggtaaaatcaaaactagtggaagtttctagaaggggaaagggataaatgaatattcatgttttgttgtgttgtgttcttcgagccagtgagagtggggaagtcgatttggtgtttagagtgtaccatcgcattttgataatgattagactcgcaaccttgaacctctctgaggttgagtttgcaaattgagaaatgattgtgataatttttgtgacgactttctctctcatattcaggctgtgtttttattttctccctgtattactttggtttttgtatcaattctcatttttaattgtctgacaaaaatgattgttcaaataacactcctcgagcaaattgtatgaatttgatccttaaattgtcatgctctcatcaatttcgtttttaaactata

SEQ ID NO: 20 - DNA, 1500 bp, Medicago truncatula
aaaagacaaattaggaagaagaaaacatttcttacctgacatcaagataaacatgggtggtcttgatcagagacttggttgcaagcacatcaacagtgacaacctcagtttttgaactgcaagagaaaaaacaacattaagaaaatgaaattaactaaaaaacagtgacacattgaagatgttagtacaaattacaaaacatgtaaaagtgtcacctctcaattccaatagaacccatgataaagtgaagtctagtagtatctatctatcaattgaagatgtacgtggcttttggattgatgaaaaacggtgagagctgaattagggtttatatagagaaaggaaaataattaaataagaaatgatggcattggcagagttgatgatgataatatataataatatatactctcatgtatctagacatgtaatgggtttatgtacaataataatttggggtgtgtactgttatggatgaatattgaatagctcccacttttgaggatatatgtgggtgcttgcttaattgcttctgcttctcaactgcgtaatgagtcagattcgttgcgtactgagtcggatcccagctgcatgattaaaaattgtctaatttaacttaattttgcaataattgttttttttagtctaatgaatataattcatctttaaatcaaataagtgatgtatgaggttcatacttcgacctttacatactacaatctaacaatcgaactatatttacggagatgtttatatacggtggcaagaatcctttatattaaatgtgaagtggtatgtgttcgaacacataattaagatacattaaagcaatgatatgttgaactctaatgttagggacaaagctattgaaaaattagaaaaatcatttgtaatttataacatcttctaatattatctaatattacctatgaagtaatattattgaagacaaatgcatgagtttgattgatgttaattaagcatgagtaattctactatttttgttgaaaatagaagaaaaaaatgcaggaaatagatggagaaccaaaattgtggatttaacaaaataggggattaaagctgtatttaacacaaaaaagtattcataatacggcttgtcaccatcgtccaccgttaaacgacatattgccatagggtggcgaaacaagtccaacctgctaggctgcctcatttagctcattatttttatgagttgaattgaagttttgaaatcgtctcatctagtttcgctctgcttaactcactaaaaaacagggcatgacaacataccaacttattttcttaatgattagacaaaacttaaactaaagtgacctaataacactcatcataactcaaatttctagtcgaccaacttgttcctacactaagcaacaaagcattatgatttttttttttgtcaagtaacctagtgactatagctcacacaatttaattgtaaaaaagtttggtgtctgaggttcgaacttcaacccctac

SEQ ID NO: 21 - DNA, 1500 bp, Medicago truncatula
ataaattaaaaaaaaaaactcagttatttgatagataatagatttataatatataagtataatagcagatggaatttacttttattttgtatttctaacatatataatattcattctcacatatcatgcatgactcttaattcgtaaaactggtagggagcaatcatttcagacaataaatttacagttgcatatagtttttaactaataaaactataacgattgagtaacatcatcacatctgttaaggagtcgggtgcacattcgcttcactacttgtttaacaaacaactacttgcaatttttattttttttgtgtgtactactactacttgcaagtttagaagtccctttcattttttcttcaactcaatataaaataaataaataaatgtatttatgtatgtatttatttatgtatatatacgctttgtgtagaagtaatttttgtttatgaaggatcttttcatgttactggattacgcgtattagtctagttcttatattagttacaccctcataggcatatctctaaggacactttgggtcatctaattctttagaacatttagttaaaaaaaaaaaactatactatagaggtgaatgagtcaaagtaaaaaaataataatattgacaaagtgaatgagttaaaaaatattgacagaggtgaaagaaattaattagcattggttatattaacgtgcccttgcaaagattaatatcaatgacaacccaaaaatttaagaggcttaaagcaaatattttagtgaggtcttttaaaataaaataaaaattaaatttgctaaacccagggtttgaaccaaacatcaagagtatttgaggcctcttcttgatgaattaaagccaggcaaacatctataataattttgtcatattatatatttataactaaattgaagttttttaggtctttttttatccaaaattttgaggcctaaaatcctaggataggttgtttaaccattgaatcggccctaattaatatctttagccagctagatcttaaggtatgtaactcttcttatatatacaaaaaataaatttttcatttaattcttttatgtcaaaaataattaataaaatatcaaattttaccctcattatccagttatacgagggtgcaccaacttaccaaatatcatcatcatcatcatatattaatattcatttcggccttaaaaactttaaacaggtataataaaagcaacatgcatatcatttctataaccaatggtaaaggtggataaattaaaggttgtatacttgcattcttaagcaatttttatagaattttttaggtcaataccaagcaggcgagcacatataagtagtaggtacattatgacttgaaatattatataagttgacaactctaacaagtaaataactatagcccagttgctatattttcattcatcaaaaaataaaagttgctacatttccattctatataaatcatccctctatagctagatactc

SEQ ID NO: 22 - DNA, 1500 bp, Medicago truncatula
agtacagtatattttcataaattatatttggtccccagttttgaacattgattaggagtcgcctttatatatattttttttgggtacaatagttgcctttgtagttttaattgacgagggataacatgtggtttaatatgcaaatatcttttccaactcagctcgaaatcggtgagaaaaactgagcagctttgccatgctataacatcagagtcacttgtagttcttgttcctatttcttactggtgtaattcttaatttatagtccattgcagttttgtctcaccaatttgcttctgctgtaccagtgcaccagtgcaccaagggtgcgattggaatctgttaaaaaaaaacacaaatataccagccttttcgggcaatttgtctgaaatttcgctgtaatattaagcacccattctacgtattagttactatatcaatgtgtttttataatattttctcatgctgtcaacgagacttcgcctatgtttctattggcggtggatttgttgtgaacgtgcgtttggagagatcttacaatttgtagcttctctgttagtggtgaaacgcaaaaacaaacacatacttcatctcctaatcttcatctgtttccactttttccgtatatattccattcaagcagtgtagatttgaactatgaatgaatatacaaaagaataatacttagcaaaagccttaaaaaaataaatatagctacttattaattaatttattgtaggttggttttggtccccagcacaaaaaagcagacaagaggaattcctcaagcaacctaccttatatatcttgtgttattttcgatcattgtacagttccgtcaagaagataccaacattgtttagtacaaaatagaagataataagttcaaggaaaagaacgaactctgaaatgatcaaaaccttttgaaagctccaacaccagcagcaacagctcaccttacatacgaaagtgaagttgtctactacaccatttttcaaagatgcaatgaaggcagcatcaatgaataattctgcaggtattaatttttttacagctaaatgttggaactttttctttcaaaaaaaatgttggaactttttacgctgtaaatatatcataattaattgtgccttgtgggcgtattgacttttccgttttgataaaattgtgggagaattgacttaataaaattgcaacgcataagaatcaattattttattttcttgaatggaagaatcaattattatttttttgaagaaggaagaatcaattatttttttatatgatttaattcatatacattattaatgtaaatattttttatattgtcaatcaattgtaatcgttgaattattaaaatcgcatgattttagttctttctttttttttcggtggaaagaaataactaattctaaaatcacacatataatttgattacaattaattgtaatttggtgacaaagtaaaacgttctataatttcgttttttttgttgaaact

SEQ ID NO: 23 - DNA, 1500 bp, Medicago truncatula
ataagatcaaaagattgttggaatatttatgccaaattaaatattggttttaaaaaataaaaaaatattgcgaaaacttactttatatatatatatatatatatatatatatatatatatatatatatatacacacactattgaaaggcaaaacttgtccctgttgcaacctgttgccattgtgacggctccttacatataggaaagccgcactaaactaacattaattctagaccctggtggcttcttagttcgagtgattattctcaaactgtgaagatgagacaccttgaacaacggcaacaaaagaaagaaaatcgatagtgttaaaaatatttgttggtgtgcttgcagtataatatttttaacgggctaagatttttaatatttatagttcaacaacgcattggctacttatgcatagtcgtgtatagaataaacacacacatacctcttgcacgcttgaaaaataacgtcatttggcaatatcactatggtttatggggaaacgagactcctcctagctacttattgaattgtaagtaaggttaaacttaagagtatacgttgagggctcgaaaggagacaagttttacggtttgagttcttggtagagcactaagcatccatccatgccttcgtgtcttgtacgttttatcaaatgctccctccatttatctttctttttcaaaagagacaaaggaatagatacataaaaaaatattttgacaagatatattcttagaaattgtggttgggtctaacacaaacccacaaaaccatttgtgaggtgatgattaccccacttataaacacattgtcagacatcacctatcagatgtgagactcttaacatttatgttgtaaaacatcaaaaatataattatatccgataaaaaaaaacaccaatataattatattaacatagagttcatgttttctatttattttctctctcttaacggtcatgccaatagtttcattagatgtattttatacaatgtccatattcgtacaatgtaaatctataatgttcaaataatacttttcaatcgatcacccatatagattgtataacattccgctcacacctcttgtaacttgagtttaagttcaaaattccacaattgacaaagtatacataagtttcattagaaattctcaatgaaaattgtgattatccataagtttacccttaattttgtagtaaacatagatgaaaaacatgttcaacctagactagactatagagagaaagaaacaagtttgattgaaagtggaccataatgggcttcactttgactaacccatgggcccaaaaaaaccgggttttgaaaacctaaaaaccgctttagtagatccaacggcagtccatagagacagataaccaaccaccaaaccaaggtcgatacatccaatcgttgtacgcctcctttgttgtcttgtttctcaatccatcataatcactatatataaacccaaaacctcactttatttctc

SEQ ID NO: 24 - DNA, 1500 bp, Medicago truncatula
gacgagggacggattaaatatgaattttttacgcgccaaaaagtaaagaaagcgtgacatcaaaacatcgacctagaaatcgaattttgagctgattttttgcaggcacctcaataaaaatacatctagaggatctgcaaagttgcgcagaaaaccgacaacgtttcgatttattttgatttttcgaccgaaacgcgcataatcgcaactttttttttcaaatgagcacccatccacgggacgaatcttgtcccccaaaaattgtaaggacctccagcgagaaattttggcaaatttgcacgcgggattcgttaattgcgaatattttaagcgtcattagccgaagaaacgaaaaagcaaacccccgtttggctcccgaattgcaaaatgccaccttgccttggcttggcttggcatgggcaggggaaggtctagtccaggccgcgaaaaaaaaatccaaaccggtatgaccttaataatacgggtgcgcaggcgcgcgcgaggaatttccgccccccgtcgtggtttggcccgagcacgtgctccccgcgagatttggcctaccacaaaaatttcgacaacgcgggcgcgcttgagatttttgcctacgaatcccctcccgacgtgatttggcccgtgcctcgtggagattttagcccacgaatcccctcccgacgtgatttggcctgtggctgcccaggccaagaacggcaagatttcccccactttcccaccaaatctcccccaaattcatcaaaagatcccaatttatcaaaaaaatttgatgaaaatggggcagatttggggggagcagcctgctggggagtcccccagccatgcccagccatgccccaacaaggtgctccccctatatatgcttgatgtgccatttccagttttgtagaaaaatgggtgaattttccctgcaggcataaagtcccgaaaaagtgacctagaggttgaattttttcacgattttttccaggcatgttaggggcataaaaacaacatctcacacaaaaaattagaattttccgacgagggacggattaaatatgaattttttacgcgtcgaaaactcaagaaaccgaagaatcaaaacatcgacctagaaatcgaactttgagctggttttttgcaggcacctcaataaaaatacatctagaggatctgcaaagtttcgtagaaaaccgacaacgtttcgatttattttgaattttcgaccgaaacgctcaaaatcgcaacttttttttcaaatgagcacccatccacgggacgaatcttgttccccaaaaattcaggcatgtttgggacatcgtaaggaccttctccgagaaattttggcaattttgcacgcgggaatcgttaactgcgaatatcttaagcgccagaaagtcaagaaagcgtgacatcaaaacatccacctagaaatcgaattttgagctggttttatgcaggcatctctataaaaataaaactcgaggatctgccaagtttcgt

SEQ ID NO: 25 - DNA, 1500 bp, Medicago truncatula
tcaatatccatttgcattaccacatctctataaatagcatcccttaacacaagaaaaagtatagaagcaattaacagaaaaatcaagtggtagaagttcaaaggaagaagacaaagaaaacatcatttgtggtatgtttcataaattcatctatgttgtttactcgtaccatgaagctttattttccttctggttggtgctacgctcactttgttaatttttattgctaattcttgtgtatattaatactattcttgtttttctttttgaagaatttaatactattattgttgttttttggcacttatacacttattatattatcattatttattcaatctaaaacttttttttattacataaagggccaaagtccgacaatttgacagtccttattttatataatcaacattttcctgatcgcaaatcgcatcgatcaattatttattttctttaaatttaggtttttttgctctttttttattttttattttttttagatagacgaaatgacaaaatcattataaactcacacacacatctgaaggtatcagattcgaacatcggttatgacatccagcctaacaatttcgacattttgccagttgaactaagactttagaattttttgctcgttttctttagctcctaatttggtcttcatatttttttaacaattatcaaatattagtagcgattagtgattacatcaaacatattatttattgatttgatcactacaatttacaaattttaaagtttaaaatttatgactatgtcaaatcttttactcccaatgcataatataaatatttcttttttcaaaaaataaaataaaataaccgattggatcaaaatctatattatggaataatcaaaattgctcttaaccatttccttcaaattaaaaacacaattattgatctaaaagagataaaaaagaataagttgttctcgataataattaaacatttctttcaaaatttactttatcaagatcaaaatcactctttgaagaaaaaaccgcaaaaaggaataagttgtcgactttattgactaagatcaaaatcactcttggaaaaaaaatcgcaaaaaggaataagttgttcttgataataattaaaaatagataattcaaacataaaaaaactaaatttttattttaaaaaaaaaacataaaaactattttaaacaatttcttatccgatcaatgacatactttactctttttctcttatatcattcaaacaactatcatctacaatcatttttagaatttttatttttttaaacaaattatgtcattgtatcaaaactattttcaataaagaggggtcgaattgttcttttctaatttattgtaagatcaaatcattctttttctaatattaatatggatactaattttggtaacatcaaattattctctttttagtcattgtaaaatccaatcatacttttaataattccattcaattaaatatttaattattaacaatt

SEQ ID NO: 26 - DNA, 1500 bp, Medicago truncatula
gtaaaaaaaataaaatatttaaagaagttgatgaagattcaaaggaagaagatgatgaaaattttcatcatatttctggattttttttcatttttaataaaaaatgagaaaaaaagatgtatatgtgcttctaagagagataaatgaccaaattgttacaaataaataagataaaggacccataatgtaatcttgtcttaattatatttaggtgttcaatcttaattaatttacactacaggattgacttataacaattaaaatttcacatcaaattcctttcatctgaatgtccttaaatttatcagttatattcataaaatttcttcttcccattttgattgttcaattttgtcgttcccaactgccgcatttttcaattttcgtgagaatcctcgtcgttcgtttgattccaaatatcttgagaattgtcgttatttccagtaccacgtcaggtatggtgacttgatttttcttttcgatttgatttttcttggttgtgaagtagttaattataacaattgagtaagtcatctgttgtactttgaggactgctagcaggtgttgccgctgtgatgttgtgggagtctgctagcatttttttggttgctgctgaaatggttatgtgatgaggcttttgttcttttgtttggacttaaaggctttgactgatagtgaggcatgtctatgcttgttatgagattttattaggttggctgcttaaataaaaaaataagtcacgatacctgacacagttctagaaacaacgacaattatcactataattggaaactgcggcagtttggaacgacaaaattgaacaatcaaaatgggaagaagaatgaaaatgaatcaagaattgatcaataggaaagaataaagaagaagaaatttgaatgaaggagatgtttttgttctcacgttcggggttttggaatggaagaaattttatgaatgtaactgatgaatttaaggacatccagatgaaagaaatttgatgtaaaattttatttattataagtcttgtggtgtaaattaattaagattgaacaccttaatatttaaaaaaaaaatcaattgttgtttaaaaaaaaaattaaataaaaattgttttgaacaagaaaattaaataaaaattaaaagtattaaaagttaccttatgattaagattaactctccttcaaaaaaaaaaaaagattaaaattaactctttttatttttagaaaaaatctactcatttatttgacatttaatgttacttatgtatattacattttgattggtttatatcatgagagagtaattaccctctaaacaagagaggataacgtaggcctcacctgttactcatgttaggttccttcacatgtgatcttaaagcagaagataacatgaccccatagttaaatttgtttgtttgtatgagaaggagagacgtgttacaccacatccataaattcataatcataacaccacatccataatcatatcatgattcct